SEQUENCE LISTING (2 sequences)

SEQ ID NO: 1 - DNA, 718 bp, homo sapien
acaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctc

SEQ ID NO: 2 - protein, 206 aa, homo sapien
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

(19) United States
(12) Patent Application Publication
(10) Pub. No.: US 2007/0281041 A1
(43) Pub. Date: Dec. 6, 2007

(54) Compositions and Methods Involving MDA-7 for the Treatment of Cancer

(75) Inventors: Rajagopal Ramesh, Sugarland, TX (US); Sunil Chada, Missouri City, TX (US)

Correspondence Address: FULBRIGHT & JAWORSKI L.L.P., 600 CONGRESS AVE., SUITE 2400, AUSTIN, TX, 78701, US

(73) Assignees: Introgen Therapeutics, Inc., Austin, TX (US); THE BOARD OF REGENTS OF THE UNIVERSITY OF TEXAS SYSTEM

(21) Appl. No.: 11/741,594

(22) Filed: Apr. 27, 2007

Related U.S. Application Data
(63) Continuation-in-part of application No. 10/791,692, filed on Mar. 2, 2004.
(60) Provisional application No. 60/796,006, filed on Apr. 28, 2006.

(51) Int. Cl. A61K 31/7052; A61K 31/166; A61K 31/517; A61K 33/24; A61K 45/00; C12N 15/00; C12N 5/02; A61P 35/00; A61K 38/02; A61K 31/7068; A61K 31/535

(52) U.S. Cl. 424/649; 435/375; 435/455; 514/12; 514/234.5; 514/266.2; 514/266.4; 514/44; 514/49; 514/617; 514/789

(57) ABSTRACT

The present invention concerns methods and compositions involving MDA-7 protein or an MDA-7-encoding nucleic acid and an EGFR inhibitor for the treatment of cancer. In certain embodiments, the invention specifically concerns a small molecule tyrosine kinase inhibitor, for example, erlotinib, as the EGFR inhibitor.

This application claims priority to U.S. provisional patent application Ser. No. 60/796,006 filed on Apr. 28, 2006, and is a continuation in part of pending U.S. patent application Ser. No. 10/791,692 filed on Mar. 2, 2004, both of which is hereby incorporated by reference.

### BACKGROUND OF THE INVENTION

1. Field of the Invention

The present invention relates generally to the fields of molecular biology and oncology. More particularly, it concerns methods and compositions for treating cancer involving a tumor suppressor, such as MDA-7, and another anti-cancer therapy, such as an EGFR tyrosine kinase inhibitor.

2. Description of Related Art

a. EGFR Tyrosine Kinase Inhibitors

Several EGFR inhibitors have been approved for use in treating cancer, including those that act by inhibiting EGFR's tyrosine kinase activity.

Erlotinib is a small molecule that inhibits the tyrosine kinase activity of the epidermal growth factor receptor (EGFR). It is sold under the name of TARCEVA®. It is an oral tablet that has recently been approved for use in treating non-small cell lung cancer and also for treating advanced pancreatic cancer in combination with gemcitabine in patients who have not been treated with chemotherapy.

Gefitinib is also a small molecule EGFR inhibitor and it has a similar chemical backbone as erlotinib. It is marketed under the name IRESSA® and it has been approved for use on patients with non-small cell lung cancer that have failed to respond to chemotherapy.

Lapatinib is another small molecule inhibitor of tyrosine kinase activity. Marketed under the name Tykerb™, lapatinib exhibits dual specificity by inhibiting tyrosine kinase activity of EGFR and the HER2 receptors. It was approved in March of 2007 by the United States Food and Drug Administration for use in patients with advanced metastatic breast cancer.

b. MDA-7

Melanoma differentiation-associated gene 7 (mda-7) encodes a 24 kDa protein and is a recently described tumor suppressor gene that induces cell death and apoptosis selectively in cancer cells, while sparing normal cells (Mhashilkar et al., 2001; Mhashilkar et al., 2003; Pataer et al., 2002).

Adenoviral overexpression of MDA-7 leads to tumor selective growth suppression and apoptosis induction in various tumor types including colorectal (Sarkar et al., 2002), breast (Mhashilkar et al., 2003), prostate (Mhashilkar et al., 2001), and lung carcinoma (Chada et al., 2004).

It was recently shown that the combination of adenoviral mediated delivery of mda-7 (Ad-mda7) and trastuzumab increased the anti-tumor activity in HER-2/neu (c-erbB2)-overexpressing breast cancer cells by decreasing phosphorylation of Akt and β-catenin (McKenzie et al., 2004).

Despite such successes, there remains a need to identify specific patient subsets that will most benefit from these procedures, and as a corollary, to identify methods which improve the chance of clinical benefit to these patients. One way in which cancer therapies may be improved is with the combination of multiple anti-cancer therapies. There are numerous examples of drugs and biologicals that, even though efficacious as individual therapies, show greatly improved clinical benefit when provided in combinations. However, it is rarely if ever clear, a priori, which combinations will provide such clinical benefits. The present invention addresses the need for new and improved treatments for cancer.

### SUMMARY OF THE INVENTION

Methods of the invention specifically include methods for treating cancer in a patient comprising providing to the patient MDA-7 in combination an EGFR inhibitor. The amount provided may be considered an “effective amount” in certain embodiments. It will be understood that “an effective amount” means that the patient is provided with both 1) MDA-7 and 2) EGFR inhibitor in an amount or amounts that leads to a therapeutic benefit. It will be understood that the patient is given an amount of MDA-7 and an amount of an EGFR inhibitor, both in amounts that are believed to contribute to the therapeutic benefit.

Aspects of the present invention concern an EGFR inhibitor, which refers to a compound or substance that inhibits EGFR activity, particularly those that directly inhibit EGFR activity (meaning the compound or substance acts directly on the EGFR polypeptide). In certain embodiments, the EGFR inhibitor is an agent that inhibits specifically the tyrosine kinase activity of EGFR. In even further embodiments, the inhibitor is a small molecule kinase inhibitor that inhibits EGFR, such as erlotinib, gefitinib, lapatinib, Zactiva™, canertinib, Tyrphostin AG 825, tyrphostin AG1318, tyrphostin RG13022, tyrphostin, erbstatin, RF14921, tyrphostin T23, tyrphostin T47, tyrphostin RG-13022, RG14620, tyrphostin AG879 and/or AG1478 (NSC693255). Other EGFR inhibitors include biological agents, which may be, in some embodiments, antibodies, such as cetuximab (ERBITUX®) ABX-EGF (panitumumab or Vectibix™), matuzumab, 806, or hR3. In certain embodiments, the EFGR inhibitor is an extracellular inhibitor, meaning it acts on the outside of an EGFR-expressing cell. EGFR inhibitors that are antibodies function extracellularly, while the small molecule inhibitors block tyrosine kinase activity in the cell. In certain embodiments, the EGFR inhibitor exhibits specificity for targets other than simply EGFR; for example, the EGFR inhibitor may also inhibit the activity of another tyrosine kinase receptor, such as the HER2 receptor. In particular other embodiments, methods of the invention involve inhibitors that are specific only to EGFR and they do not include a dual acting inhibitor.

Embodiments of the invention include providing a patient with both MDA-7 and an EGFR inhibitor in methods of the invention. The term “provide” is used according to its ordinary and plain meaning: “to supply or furnish for use” (Oxford English Dictionary). It is contemplated that cancer cells of the patient or cells adjacent to cancer cells of the patient are exposed to MDA-7 and the EGFR inhibitor. MDA-7 exerts a bystander effect and consequently, a cell adjacent to a cancer cell may express MDA-7 and provide it to a cancer cell. Aspects of the invention include administering to a patient, compositions providing MDA-7 and/or an EGFR inhibitor to the patient. Embodiments of the invention include methods of administering a combination of MDA-7 and an EGFR inhibitor that provides a synergistic therapeutic effect with respect to treating cancer cells. “Synergistic” indicates that the therapeutic effect is greater than would have been expected based on adding the effects of each agent applied as a monotherapy.

In certain embodiments, MDA-7 is provided to a cell(s) by administering to the cells an expression construct encoding MDA-7, which then expresses MDA-7 in the cell. In particular embodiments the expression construct is a viral vector. In further embodiments, the viral vector is an adenovirus vector containing a nucleic acid sequence encoding MDA-7.

MDA-7 may be provided to the patient by administering to the patient a composition that includes purified MDA-7 protein. In certain embodiments, the method includes subjecting the patient to radiotherapy, chemotherapy, and/or surgical resection of premalignant or malignant lesion.

The term “purified” means that MDA-7 protein was previously isolated away from other proteins and that the protein is at least about 95% pure prior to being formulated in the composition. In certain embodiments, the purified MDA-7 protein is about or is at least about 95, 96, 97, 98, 99, 99.1, 99.2, 99.3, 99.4, 99.5% pure or more, or any range derivable therein. Moreover, it is contemplated that purified MDA-7 protein is active, meaning it is capable of inducing apoptosis. The purified MDA-7 protein can also be qualified in terms of activity such that it is about, at least about, or at most about 50, 55, 60, 65, 70, 75, 80, 85, 90, 95% or more (or any range derivable therein) as active (measured by apoptotic activity) as an equivalent amount of MDA-7 that is not purified (such as prepared by recombinant means).

In other embodiments, a composition contains a compound that can lead to an active MDA-7 polypeptide or EGFR inhibitor in the patient or in cells of the patient, such as a prodrug compound or a nucleic acid encoding the polypeptide.

A patient can be any animal, including a human, having, suspected of having, or at risk or heightened risk of having cancer and undergoes treatment for such. In many embodiments of the invention, a patient is a mammal, specifically a human. The patient/subject can be one known or suspected of being free of a particular disease or health-related condition at the time the inventive compositions and/or methods are administered. The subject, for example, can be a subject with no known disease or health-related condition (i.e., a healthy subject). In some embodiments, the subject is a subject at risk of developing a particular disease or health-related condition. For example, the subject or the subject's relatives may have a history of cancer, who is at risk of developing a cancer. Alternatively, the subject may have undergone failed cancer therapy. The subject may be a subject at risk of developing a recurrent cancer because of a genetic predisposition or as a result of past chemotherapy. Alternatively, the subject may be a subject with a history of successfully treated cancer who is currently disease-free, but who is at risk of developing a second primary tumor. For example, the risk may be the result of past radiation therapy or chemotherapy that was applied as treatment of a first primary tumor. In some embodiments, the subject may be a subject with a first disease or health-related condition, who is at risk of development of a second disease or health-related condition. In some embodiments, methods may involve identifying a patient in need of such treatment. A patient may be identified, for example, based on taking a patient history, having one or more tests done to determine that the patient has cancer or a tumor, operating on the patient or taking a biopsy. In certain embodiments the patient may be identified as having a cancer that overexpresses EGFR. In additional embodiments, the cancer or tumor is evaluated to determine whether methods of the invention are an appropriate treatment. This may include, for example, evaluating whether the cancer is an EGFR-overexpressing cancer.

A cancer can be any type of cancer. For example, the cancer may be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder cancer. In certain embodiments, the cancer involves epithelial cancer cells. In specific embodiments, the cancer is breast cancer, lung cancer, or prostate cancer. A cancer may involve an unresectable or resectable tumor. In some embodiments, the cancer appears resistant to radiotherapy, chemotherapy, and/or immunotherapy (such as trastuzumab), or to any of the agents discussed herein, but as a monotherapy (compared to the combination of MDA-7 plus an EGFR inhibitor). Furthermore, the cancer may involve a metastasized or second tumor, though in some embodiments, it concerns only one or more primary tumors. It is further contemplated that the methods and compositions of the invention can be implemented for inhibiting metastasis of a tumor or preventing the further growth of a tumor, as well as for reducing or eliminating a tumor or cancer.

The present invention can be used to induce apoptosis in cells. It is contemplated that this can be employed in methods and compositions for treating cancer. The cancer can be any of the following types of cancer: melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, bronchial, lymphoma, brain, colon, or bladder. In certain embodiments, the cancer involves epithelial cancer cells. In specific embodiments the cancer is lung cancer, such as non-small cell lung cancer. In other embodiments, the cancer is pancreatic cancer. In still other embodiments the cancer is glioma. In specific embodiments, the cancer is breast cancer. In the case of breast cancer, the patient can be HER-2/neu negative or the patient can be HER-2/neu positive. Thus, the treatment can be independent of HER-2/neu status of the patient. In other embodiments a cancer can be identified as overexpressing EGFR.

“Treatment” and “treating” refer to administration or application of an agent, drug, compositions, or remedy to a subject, or performance of a procedure or therapeutic action on a subject for the purpose of obtaining a therapeutic benefit against a disease or health-related condition.

A “disease” or “health-related condition” can be any pathological condition of a body part, an organ, or a system resulting from any cause, such as infection, genetic defect, and/or environmental stress. The cause may or may not be known. Examples of such conditions include, but are not limited to, premalignant states, dysplasias, cancer, and other hyperproliferative diseases. A cancer, for example, may be a recurrent cancer or a cancer that is known or suspected to be resistant to conventional therapeutic regimens and standard therapies.

The term “therapeutic benefit” used throughout this application refers to anything that promotes or enhances the well-being of the subject with respect to the medical treatment of a condition, which includes, but is not limited to, treatment of pre-cancer, dysplasia, cancer, and other hyperproliferative diseases. A list of nonexhaustive examples of therapeutic benefit includes extension of the subject's life by any period of time, decrease or delay in the neoplastic development of the disease, decrease in hyperproliferation, reduction in tumor growth, delay of metastases or reduction in number of metastases, reduction in cancer cell number or tumor cell proliferation rate, decrease or delay in progression of neoplastic development from a premalignant condition, and a decrease in pain to the subject that can be attributed to the subject's condition.

“Prevention” and “preventing” are used according to their ordinary and plain meaning to mean “acting before” or such an act. In the context of a particular disease or health-related condition, those terms refer to administration or application of an agent, drug, or remedy to a subject, or performance of a procedure or modality on a subject for the purpose of blocking the onset of a disease or health-related condition. In certain embodiments of the present invention, the methods involving delivery of MDA-7 protein or a nucleic acid encoding the protein to prevent a disease or health-related condition in a subject. An amount of a pharmaceutical composition that is suitable to prevent a disease or condition is an amount that is known or suspected of blocking the onset of the disease or health-related condition. The invention contemplates that MDA-7 may be provided to a subject in addition to at least one other agent, such as an EGFR inhibitor.

Accordingly, in some embodiments, MDA-7 is provided to the patient by administering to the patient a composition comprising a nucleic acid having a sequence encoding MDA-7 polypeptide, wherein the MDA-7 polypeptide is expressed in the patient. It is contemplated that the MDA-7 encoding nucleic acid sequence is under the control of a promoter capable of providing expression in the patient. The promoter can be constitutive, tissue-specific, repressible, or inducible. In certain embodiments, the promoter is the CMV IE promoter. In additional embodiments, an enhancer is included.

A vector, including an expression construct, can be employed in methods of the invention to provide MDA-7 to a patient. In certain embodiments, the vector is a viral vector. If a viral vector is used, in some embodiments the vector is formulated with protamine. In certain embodiments, a vector is formulated with one or more lipids. Embodiments include a lipid formulation comprising a DOTAP:cholesterol (or derivative thereof) (DOTAP:chol) formulation. In some embodiments, an anti-inflammatory agent is administered before, during, or after administration of the MDA-7. Compositions administered to a patient are typically in a pharmaceutically acceptable formulation. Any lipid or lipid composition suitable for pharmaceutical administration is contemplated by the present invention. In certain embodiments, the composition is further defined as comprising a liposome. Any liposome suitable for pharmaceutical administration is contemplated for inclusion in the methods of the present invention.

Viral vectors that can be used are adenovirus, adeno-associated virus, herpesvirus, lentivirus, retrovirus, and vaccinia virus. In specific embodiments, the vector is an adenovirus vector, which can be replication-deficient. In this case, it is contemplated that about 10⁹ to about 10¹³ viral particles (vp) or plaque forming units (pfu) are administered to the patient either per administration (patient/administration) or per day (average daily dose). Such doses include about, at least about, or at most about 10⁹, 10¹⁰, 10¹¹, 10¹², or 10¹³ vp or pfu (or any range derivable therein), which may be the amount given per administration or per day or per treatment cycle. Alternatively, dosage for a patient may be expressed as between about 10⁷ to about 10¹¹ vp/kg with the above doses being administered.

Compounds and compositions (including MDA-7 nucleic acid and/or protein compositions) may be administered to a patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, intrathecally, orally, topically, locally, by inhalation, by injection, by infusion, by continuous infusion, by localized perfusion bathing target cells directly, via a catheter, or via a lavage. It is contemplated that a combination of routes of administration may be employed. For instance, MDA-7 may be provided by one route while the EGFR inhibitor is provided by another route. Alternatively, it is contemplated that one dose of either MDA-7 or the EGFR inhibitor is administered to the patient while another dose is administered to the patient in a different manner. In specific embodiments, an EGFR inhibitor is ingested by the patient, such as in a tablet formulation, or administered intravenously to the patient.

In certain embodiments, it is contemplated that a compound(s) or composition(s) is directly injected into or directly administered to a tumor. Alternatively or additionally, a compound(s) or composition(s) is applied or administered to a residual tumor bed.

MDA-7 (as a protein or nucleic acid encoding the protein) can be provided to a patient the following number of times or at least the following number of times: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more times as part of a therapy. It is specifically contemplated that a patient is provided with MDA-7 and the EGFR inhibitor more than once as part of the patient's cancer treatment.

Moreover, the present invention can be used to prevent cancer or to treat pre-cancers or premalignant cells, including metaplasias, dysplasias, and hyperplasias. It may also be used to inhibit undesirable but benign cells, such as squamous metaplasia, dysplasia, benign prostate hyperplasia cells, hyperplastic lesions, and the like. The progression to cancer or to a more severe form of cancer may be halted, disrupted, or delayed by methods of the invention involving MDA-7 in combination with an EGFR inhibitor as discussed herein.

In specific embodiments, the present invention concerns methods of treating cancer in which a patient with cancer is provided MDA-7 and an EGFR inhibitor. Other methods of the invention concern treating breast cancer in a patient comprising administering to the patient a i) an adenovirus vector comprising a nucleic acid sequence encoding MDA-7, wherein the nucleic acid sequence is under the control of a promoter capable of being expressed in the patient; and, ii) a EGFR inhibitor.

It is contemplated that in some embodiments of the invention, a patient is provided with the EGFR inhibitor by administering a EGFR inhibitor directly to the patient. Moreover, more than one EGFR inhibitor may be employed, such as a combination of 2, 3, or 4 such inhibitors. The EGFR inhibitor may be any EGFR inhibitor known to those of ordinary skill in the art. For example, the inhibitor may be a DNA, RNA, an oligonucleotide, a ribozyme, a protein, a polypeptide, a peptide, an antibody, an oligosaccharide, or small molecule. In particular embodiments, the EGFR inhibitor is an antibody, such as an antibody directed against EGF or a EGFR. In more particular embodiments, the antibody is a monoclonal antibody, such as a monoclonal antibody that specifically binds EGF or EGFR. In a more particular embodiment, the EGFR inhibitor is erlotinib or gefitinib. In some embodiments, the EGFR inhibitor is a small molecule. Examples of such small molecules include small molecule tyrosine kinase inhibitors of EGFR. In particular embodiments, the EGFR inhibitor is a ribozyme or siRNA that specifically targets EGFR mRNA or its expression/translation. In further particular embodiments, the EGFR inhibitor is a soluble EGFR.

In some embodiments, methods may involve identifying a patient in need of such treatment. Aspects of the invention include methods for treating cancer in a patient comprising a) providing to the patient MDA-7; and b) administering to the patient a small molecule EGFR tyrosine kinase inhibitor. Typically, MDA-7 is provided to the patient by administering to the patient a composition comprising a nucleic acid having a sequence encoding a MDA-7 polypeptide, wherein the MDA-7 polypeptide is expressed in the patient. In certain aspects the nucleic acid is comprised in a vector, in particular a viral vector, and more particularly an adenovirus vector. In still further aspects the adenovirus vector can be formulated with protamine. The viral vector can be administered to a patient in an amount of about 10⁸, 10⁹, 10¹⁰ to about 10¹¹, 10¹², 10¹³ viral particles per administration. Aspects of the invention include an MDA-7 nucleic acid composition comprising one or more lipids. The compositions of the invention can comprise DOTAP and cholesterol, or a derivative thereof. Embodiments of the invention include methods were MDA-7 is provided to a patient by administering to the patient a composition comprising purified MDA-7 protein.

MDA-7 can be provided prior to, concurrently with, or following administration of the inhibitor to the patient. Also, the inhibitor can be administered prior to, concurrently with, or following provision MDA-7 to the patient. In certain aspects MDA-7 is provided to the patient via one route of administration and the inhibitor is administered to the patient via a different route. Further aspects of the invention include providing MDA-7 to the patient via the same or different route of administration as the inhibitor, and vice versa. In certain embodiments the MDA-7 is provided to the patient with 1, 2, 3, 4, 5, 6, 7, 8, 9, 10 or more days or weeks of administering the inhibitor. In still a further embodiment, the MDA-7 is provided to the patient within 2, 4, 6, 8, 10, 12, 14, 16, 18, 20, 22, 24, 26, 28, 30, 32, 34, 36, 38, 40, 42, 44, or 48 hours of administering the inhibitor. Aspects of the invention include administering MDA-7 and/or an inhibitor multiple times. Further aspects include providing given multiple courses of therapy with MDA-7 and the inhibitor. The methods can further comprise treating the patient with another anti-cancer therapy including, but not limited to chemotherapy or radiotherapy. In particular aspects the chemotherapy is platinum-based chemotherapy. Still further aspects include where platinum-based chemotherapy involves one or more of carboplatin, paclitaxel, gemcitabine, or cisplatin. The methods can include a chemotherapy that involves more than 1, 2, 3 or more drugs or agents. The chemotherapy can include a combination of carboplatin and paclitaxel or a combination of gemcitabine and cisplatin. In certain embodiments the patient has or will undergo tumor resection. Aspects of the invention include identifying a patient in need of the treatment. Identifying a patient in need of the treatment can comprise taking a patient history regarding previous cancer treatment. In certain aspects a patient has failed previous cancer therapy or has a recurrent or metastasized cancer. A patient has, is suspected of having, or has had cancer of the lung, prostate, liver, pancreas, bladder, breast, ovary, gastric, colon, head and neck, esophagus, synovium, brain, or bronchus. Typically, compositions of the invention are included in a pharmaceutically acceptable composition.

As set forth above, the MDA-7 may be provided to the patient by administering to the patient a composition that includes a nucleic acid having a sequence encoding MDA-7 polypeptide, wherein the MDA-7 polypeptide is expressed in the patient. In particular embodiments, the composition is a pharmaceutically acceptable composition. Alternatively, the MDA-7 may be provided to the patient by administering to the patient a purified MDA-7 protein composition, as discussed above. In certain particular embodiments, the composition is a pharmaceutically acceptable composition.

It is contemplated that in some embodiments a patient is provided with MDA-7 and an EGFR inhibitor in a single composition. Compositions to be administered to a patient include compositions of the present invention, which are disclosed herein. Furthermore, in some embodiments, the patient is provided with a composition comprising the EGFR inhibitor and either i) purified MDA-7 protein or ii) a nucleic acid having a sequence encoding MDA-7.

In other embodiments, MDA-7 and an EGFR inhibitor are provided separately to the patient. In such cases, it is contemplated that the patient is provided with one agent and the other agent is provided or administered within 1, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 day and/or 1, 2, 3, 4, 5, 6, 7, 8, 9, 10, 11, 12 weeks, or any range derivable therein. In certain embodiments, it is contemplated that the patient is provided with MDA-7 within 24 hours of being provided with the EGFR inhibitor. In other embodiments, the patient is provided with the MDA-7 within 2 hours of being provided with the EGFR inhibitor. In some embodiments, the patient is provided with the MDA-7 prior to being provided with the EGFR inhibitor, while in other embodiments, the patient is provided with the EGFR inhibitor prior to being provided with the MDA-7. Furthermore, it is contemplated that a patient may take or be administered EGFR inhibitor intermittently throughout the course of treatment with MDA-7. For example, it is contemplated that a patient may undergo MDA-7 therapy for a six week period. During that time, the patient may take, for example, a EGFR inhibitor throughout the six-week period, such as at least on a daily or weekly basis. Therefore, it is contemplated that a patient may take or be provided with an EGFR inhibitor within 24 hours (or any time period specified above) of being provided MDA-7 and that MDA-7 may be provided more than once. Accordingly, the patient will have taken or be provided with an EGFR inhibitor within any of the specified amount of times discussed above with respect to each time that MDA-7 is provided to the patient (either as a protein or a nucleic acid encoding the protein). It is furthermore contemplated that within any time period specified above, the EGFR inhibitor may be taken or be provided multiple times. For example, a patient may take three doses of an EGFR inhibitor within 24 hours of being provided with MDA-7. Consequently, a patient may take or be provided an EGFR inhibitor 1, 2, 3, 4, 5, 6, 7, 8, 9, 10, 11, 12, 13, 14, 15, 16, 17, 18, 19, 20 or more individual times, or any range derivable therein, within a specified time period of being provided the MDA-7. The EGFR inhibitor may be provided systemically during or throughout a course of therapy with MDA-7.

In some embodiments the patient is subjected to radiotherapy after being provided MDA-7 and an EGFR inhibitor each at least once. In further embodiments a patient is subjected to a sub-lethal dose of radiotherapy. The term “sub-lethal dose” refers to an amount of radiation given to a patient in a single session that is less than a lethal amount (i.e., amount that causes cell to die) for cells of the patient exposed to the radiation. It is contemplated that a sub-lethal dose is lower than the dose currently given to a cancer patient with similar characteristics (referring to, e.g., stage of cancer, size of tumor, prognosis, etc.) who are not first provided with radiosensitization treatment. It is contemplated that the radiosensitization treatment may preceed exposure to radiation by about, at least about, or at most about 5, 10, 15, 20, 25, 30, 35, 40, 45, 50, 55, 60 minutes, and/or, 2, 3, 4, 5, 6, 7, 8, 9, 10, 11, 12, 13, 14, 15, 16, 17, 18, 19, 20, 21, 22, 23, 24 hours, and/or 1, 2, 3, 4, 5, 6, 7 days, or more, or any range derivable therein.

In certain embodiments of the invention, methods also include subjecting the patient to radiotherapy and/or chemotherapy. In other embodiments, the patient is subjected to immunotherapy. In other particular embodiments, methods also involve resecting all or part of a tumor from the patient. It is contemplated that multiple tumors may be removed (whole or part). In each of these cases, MDA-7 and/or an EGFR inhibitor can be provided, before, during or after the other cancer therapy. In certain embodiments, MDA-7 and/or an EGFR inhibitor is provided to the patient after tumor resection, such as by administering a composition with one or multiple agents to at least the resulting tumor bed.

Other embodiments of the invention include providing a different tumor suppressor in place of MDA-7 in embodiments of the invention. Other tumor suppressors include, but are not limited to p53, FUS1, C-CAM, FHIT, DCC, Rb, and PTEN. As such, the protein or a nucleic acid encoding the utmor suppressor may be employed as discussed above.

The present invention also concerns pharmaceutical compositions. In some embodiments, there is a pharmaceutical composition that includes a) an EGFR inhibitor; and b) purified and active MDA-7 protein or a nucleic acid having a sequence encoding MDA-7 polypeptide. It is contemplated that in embodiments involving a MDA-7 encoding nucleic acid, the nucleic acid may be an adenovirus vector. Pharmaceutical compositions may contain one or more of EGFR inhibitors.

In certain embodiments, the pharmaceutical composition includes a nucleic acid having a sequence encoding MDA-7 polypeptide. In particular embodiments, the nucleic acid encoding MDA-7 polypeptide is an adenovirus vector. The pharmaceutical composition may include a nucleic acid having a sequence encoding an siRNA or an antisense RNA designed to inhibit EGFR. The nucleic acid encoding the siRNA or an antisense RNA designed to inhibit EGFR may be an adenovirus vector. In a particular embodiment, the pharmaceutical composition includes (a) a first adenovirus vector having a nucleic acid sequence encoding MDA-7, wherein the nucleic acid sequence is operably coupled to a first promoter sequence; and (b) a second adenovirus vector having a sequence encoding an siRNA or an antisense RNA designed to inhibit EGFR., wherein the nucleic acid sequence encoding the siRNA or an antisense RNA designed to inhibit EGFR is operably connected to a second promoter.

In some embodiment, the pharmaceutical composition includes an adenovirus vector having a first nucleic acid sequence encoding MDA-7 and a second nucleic acid sequence encoding an siRNA or an antisense RNA designed to inhibit EGFR. The first nucleic acid sequence and the second nucleic acid sequence may or may not be operably connected to one or more common promoters.

The present invention also pertains to methods of treating or preventing cancer in a patient, that include administering to the patient a pharmaceutically acceptable composition comprising a polynucleotide encoding an MDA-7 protein and a lipid. The cancer can be any of those cancers set forth above. In certain embodiments, the patient is a patient with lung cancer. For example, the lung cancer may be a non-small cell lung, small-cell lung, or a metastatic lung cancer (cancer that has spread outside the confines of the lung). Treatment of the primary lung cancer may be effected, in addition to treatment of a secondary tumor from the lung cancer. In further embodiments, the method is further defined as a method of treating metastatic lung cancer in a subject.

In some embodiments, the patient has a history of cancer that has been successfully treated with chemotherapy, radiotherapy, chemotherapy, immunotherapy, and/or gene therapy, but the patient is later diagnosed with cancer again.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Cell viability of lung tumor cells (H1299) after erlotinib, Ad-luc (luciferase), Ad-mda7, Ad-luc plus erlotinib or Ad-mda7 plus Tarceva after 72 hours. H1299 cells were treated with PBS (phosphated buffered saline) as control. Ad-luc or Ad-mda7 was administered at an MOI of 2000 viral particles per cell. Erlotinib, dissolved in DMSO, was administered at a dose of either 2.5 or 5 μm per well (cells initially plated in 6 well plates at 5×10⁴ cells/well). The viability was measured by counting after trypan blue exclusion assay. The combination of erlotinib and Ad-mda7 resulted in a significant decrease cell viability (P<0.05) after an incubation of 72 hours as compared to the control cells treated with Ad-luc plus erlotinib.

FIG. 2. Light microscopy of cell viability of lung tumor cells (H1299) after erlotinib, Ad-luc (luciferase), Ad-mda7, Ad-luc plus erlotinib or Ad-mda7 plus erlotinib after 72 hours.

FIG. 3. Cell viability of prostate tumor cells (LNCaP) after erlotinib, Ad-luc (luciferase), Ad-mda7, Ad-luc plus erlotinib or Ad-mda7 plus erlotinib after 72 hours. H1299 cells treated with PBS (phosphated buffered saline) alone or with Ad-luc served as controls. Ad-luc or Ad-mda7 was administered at an MOI of 2000 viral particles per cell. Erlotinib, dissolved in DMSO, was administered at a dose of either 2.5 or 5 μm per well (cells initially plated in 6 well plates at 5×10⁴ cells/well). The viability was measured by counting after trypan blue exclusion assay. The combination of erlotinib and Ad-mda7 resulted in a significant decrease cell viability (P<0.05) after an incubation of 72 hours as compared to the control cells treated with Ad-luc plus Tarceva.

FIGS. 4A-4B. Trandsuction efficiency of adeoviral vectors in lung tumor cells (H1299) after Ad-gfp (green fluorescent protein), Ad-gfp plus DMSO or Ad-gfp plus erlotinib after 72 hours. H1299 cells treated with PBS (phosphated buffered saline) alone served as a control. H1299 cells lines (5×10⁴ cells/well in 6-well plates) were transduced with Ad-GFP at 50 or 100 vp/cell in the presence of either DMSO alone or erlotinib dissolved in DMSO (5 μM per well). Harvested cells were subjected to FACS analysis to determine the percentage of mean fluorescent intensity (A) and GFP positive cells (B).

FIG. 5. Western blot analysis of lung tumor cells (H1299) treated with Ad-luc (luciferase), Ad-luc plus erlotinib, Ad-mda7 or Ad-mda7 plus erlotinib after 24, 48 or 72 hours. H1299 cells treated with PBS (phosphated buffered saline) alone served as a control. Ad-luc or Ad-mda7 was administered at an MOI of 2000 viral particles per cell. Erlotinib, dissolved in DMSO, was administered at a dose of 5 μm per well (cells initially plated in 6 well plates at 5×10⁴ cells/well). The western blot was probed with primary antibodies to Caspase-3, MDA-7, Src and EGFR. Protein signals were detected using enhanced chemiluminescence (ECL). Increased inhibition of EGFR phosphorylation in cells treated with the combination of erlotinib and Ad-mda7 indicated a synergistic effect. Cleaved caspase-3 indicated a correlation with the enhanced growth inhibition of those cells treated with Ad-mda7 plus erlotinib.

FIG. 6. Cell Viability Assay. The effect of lapitinib and Ad-mda7 on HER2 overexpressing (MDA-MB-453 and EGFR (HER1) overexpressing (MDA-MB-468) human breast cancer cell lines was evaluated by cell counting after trypan blue (Invitrogen Co., Carlsbad, Calif.) exclusion assay. MDA-MB-453 and MDA-MB-468 cell lines (1×10⁵ cells/well in 6-well plates) were treated with PBS or transduced with either Ad-luc or Ad-mda7 at an MOI of 1000 viral particles (vp)/cell. Lapatinib, dissolved in DMSO, was administered to both transduced and PBS treated cell cultures at a dose of 5 μg/ml per well (2 ml of media per well). Cells not administered lapatinib (either PBS treated or transduced with either adenoviral vector) served as controls. Seventy-two hours after adenoviral vector transduction, cells were trypsinized and an aliquot suspended 1:1 volume with 0.4% trypan blue. Total cell numbers and cell viability counts were assessed using a hemocytometer by light microscopy.

FIG. 7. Apoptosis Assay. MDA-MB-468 cells (1×10⁵ cells per well in a 6 well plate) were treated with either Ad luc, Ad-luc plus lapatinib, Ad-mda7 or Ad-mda7 plus lapatinib at an MOI of 1000 vp per cell. Lapatinib was administered at a concentration of 300 nM per well. Cells were harvested post treatment at day 2, 3 and 4. Hervested cells were fixed, stained with propidium iodide and analyzed via floy cytometry.

FIG. 8. Cell Viability Assay. The effect of cetuximab and Ad-mda7 on human non-EGFR expressing (MCF-7) and EGFR overexpressing (MDA-MB-468) human breast cancer cell lines was evaluated by cell counting after trypan blue (Invitrogen Co., Carlsbad, Calif.) exclusion assay. Briefly, MCF-7 and MDA-MB-468 cell lines (1×10⁵ cells/well in 6-well plates) were treated with PBS or transduced with either Ad-luc or Ad-mda7 at an MOI of 1000 viral particles (vp)/cell. Cetuximab, dissolved in PBS, was administered to both transduced and PBS treated cell cultures at a dose of 5 μg/ml per well (2 ml of media per well). Cells not administered cetuximab (either PBS treated or transduced with either adenoviral vector) served as controls. Seventy-two hours after adenoviral vector transduction, cells were trypsinized and an aliquot suspended 1:1 volume with 0.4% trypan blue. Total cell numbers and cell viability counts were assessed using a hemocytometer by light microscopy.

FIG. 9. Western Blot Assay. Expression of Human EGF receptors varies in breast cancer cell lines. MDA-MB-231 is a breast cancer cell line which overexpresses EGFR and does express some HER2. MDA-MB-453 overexpresses HER2 and exhibits minimal expression of EGFR. MDA-MB-468 overexpresses EGFR and does not express HER2. BT-474 is a breast cancer cell line which overexpresses both EGFR and HER2. MCF-7 does not overexpress either EGFR or HER2.

FIG. 10. Cell Viability Assay. Cell viability of both the EGFR overexpressing cell lines (MDA-MB-468) and the HER2 overexpressing cell line (MDA-MB-452) decreased as cetuximab dosage increases. However, the slight increase in cell death in HER2 overexpressing cells may have been the result of some EGFR expression in these cells or some cross reactivity of cetuxumab with HER2. No increase in cell death was seen from the MCF-7 cell line, which does not express either EGFR or HER2.

FIG. 11. Western Blot Analysis. Western blot analysis demonstrates that MDA-7 strongly inhibits the phosphorylation of EGFR as dosage increases. Likewise, MDA-7 inhibits the phosphorylation of HER2 in a dosage dependent manner.

FIG. 12. Western Blot Analysis. MDA-MB-453 and MDA-MB-468 cell lines were treated with MDA-7 protein in escalating dosage of 3.8, 7.7, 15.0 and 30.0 ng/ml. 48 hours following treatment cells were harvested and subjected to western blot. As indicated in the figure, MDA-7 protein, and not adenoviral vector is the causative agent responsible for the inhibition of phosphorylation of HER2 and EGFR.

FIG. 13. Western Blot Analysis. MDA-MB-453 cells (1×10⁵ cells/well in 6-well plates), which overexpress HER2 were treated with PBS or Cetuximab, dissolved in PBS (5 μg/ml per well), or lapatinib dissolved in DMSO (5 μg/ml per well) (2 ml of media per well) or a combination of each. Cells were optionally treated with Ad-mda7 (MOI 1000 vp/cell). As seen in the figure, the combinations of lapatinib+Ad-mda7, cetuxumab+Ad-mda7, and lapatinib+cetuximab+Ad-mda7 resulted in a greater inhibition of phosphorylated ERK than did treatment with any of these reagents alone.

### DESCRIPTION OF ILLUSTRATIVE EMBODIMENTS

Compositions and methods of the invention include compositions and methods for treating cancer in a patient comprising providing to the patient MDA-7 in combination an EGFR inhibitor. The amount provided may be considered an “effective amount” in certain embodiments. It will be understood that “an effective amount” means that the patient is provided with both 1) MDA-7 and 2) EGFR inhibitor in an amount or amounts that leads to a therapeutic benefit. It will be understood that the patient is given an amount of MDA-7 and an amount of an EGFR inhibitor, both in amounts that are believed to contribute to the therapeutic benefit.

I. MDA-7 Compositions

The compositions and methods of the present invention employ MDA-7 polypeptides and nucleic acids encoding such polypeptides. MDA-7 is a tumor suppressor that has been shown to suppress the growth of cancer cells that are p53-wild-type, p53-null and p53-mutant. Also, the observed upregulation of the apoptosis-related B gene in p53 null cells indicates that MDA-7 is capable of using p53-independent mechanisms to induce the destruction of cancer cells.

Mda-7 mRNA has been identified in human PBMC (Ekmekcioglu et al., 2001), and no cytokine function of human MDA-7 protein was reported. MDA-7 has been designated as IL-24 based on the gene and protein sequence characteristics (NCBI database accession XM_{—}001405). The murine MDA-7 protein homolog FISP (IL-4-Induced Secreted Protein) was reported as a Th2 specific cytokine (Schaefer et al., 2001). Transcription of FISP is induced by TCR and IL-4 receptor engagement and subsequent PKC and STAT6 activation as demonstrated by knockout studies. Expression of FISP was characterized but no function has been attributed yet to this putative cytokine (Denkert et al., 2004). The rat MDA-7 homolog C49a (Mob-5) is 78% homologous to the mda-7 gene and has been linked to wound healing (Soo et al 1999; Zhang et al., 2000). Mob-5 was also shown to be a secreted protein and a putative cell surface receptor was identified on ras transformed cells (Zhang et al., 2000). Therefore, homologues of the MDA-7 gene and the secreted MDA-7 protein are expressed and secreted in various species. However, no data has emerged to show MDA-7 has cytokine activity. Such activity has ramifications for the treatment of a wide variety of diseases and infections by enhancing immunogenicity of an antigen.

The human mda-7 cDNA (SEQ ID NO:1) encodes an evolutionarily conserved protein of 206 amino acids (SEQ ID NO:2) with a predicted size of 23.8 kDa. The deduced amino acid sequence contains a hydrophobic stretch from about amino acid 26 to 45, which has characteristics of a signal sequence. A combination of structural data, homology to known cytokines, chromosomal localization, a predicted N-terminus secretion signal peptide, and evidence of its regulation of cytokine secretion, all support classification of MDA-7/IL-24 as a IL-10 family cytokine (see Chada et al., 2004 review). A 49 amino acid leader sequence identifies it as a secreted protein; recent studies confirm this and report that Ad-mda7 transduced cells release high levels of a 40 kDa form of the MDA-7 protein, which can bind to heterodimeric receptors IL-20R1/IL-20R2 and IL-22R2/IL-20R1. The intracellular form of the protein (23-30 kDa) is cleaved, and extensively modified (primarily by glycosylation) before its release into the extracellular compartment (see Chada et al, 2004 review, which is hereby incorporated by reference). In certain embodiments of the invention, the MDA-7 used in the invention lacks the 49 amino acid leader. The leader may be replaced with a heterologous signal or leader sequence. In further embodiments, the sequence targets the protein to the endoplasmic reticulum (ER) and thus, is an ER-leader sequence. Such sequences are known to those of skill in the art.

The expression of MDA-7 is inversely correlated with melanoma progression as demonstrated by increased mRNA levels in normal melanocytes as compared to primary and metastatic melanomas as well as decreased MDA-7 expression in early vertical growth phase melanoma cells selected for enhanced tumor formation in nude mice. Reports indicate that MDA-7 is an IL-10 family cytokine with tumor cell apoptotic activity and that the cytotoxic effects it induces are specific to tumor cells (see Chada et al., 2004 review). Several studies have investigated the signal transduction pathways that mediate the apoptotic activity of mda-7. These appear to be multiple, cell-type specific, and include effects induced by the intracellular form of the protein, and by the secreted form (bystander effect) (see U.S. patent application Ser. No. 10/791,692, which is incorporated by reference). Additional information and data regarding MDA-7 can be found in U.S. patent application Ser. Nos. 09/615,154, 10/017,472, 10/378,590, 10/791,692, and 11/349,727, all of which are incorporated by reference in their entireties.

Additional studies have shown that elevated expression of MDA-7 suppressed cancer cell growth in vitro and selectively induced apoptosis in human breast cancer cells as well as inhibiting tumor growth in nude mice (Jiang et al., 1996 and Su et al., 1998). Jiang et al. (1996) report findings that MDA-7 is a potent growth suppressing gene in cancer cells of diverse origins including breast, central nervous system, cervix, colon, prostate, and connective tissue. A colony inhibition assay was used to demonstrate that elevated expression of MDA-7 enhanced growth inhibition in human cervical carcinoma (HeLa), human breast carcinoma (MCF-7 and T47D), colon carcinoma (LS174T and SW480), nasopharyngeal carcinoma (HONE-1), prostate carcinoma (DU-145), melanoma (HO-1 and C8161), glioblastome multiforme (GBM-18 and T98G), and osteosarcoma (Saos-2).

Su et al. (1998) reported investigations into the mechanism by which MDA-7 suppressed cancer cell growth. The studies reported that ectopic expression of MDA-7 in breast cancer cell lines MCF-7 and T47D induced apoptosis as detected by cell cycle analysis and TUNEL assay without an effect on the normal HBL-100 cells. Western blot analysis of cell lysates from cells infected with adenovirus mda-7 (“Ad-mda7”) showed an upregulation of the apoptosis stimulating protein BAX. Ad-mda 7 infection elevated levels of BAX protein only in MCF-7 and T47D cells and not normal HBL-100 or HMEC cells. These data lead the investigators to evaluate the effect of ex vivo Ad-mda7 transduction on xenograft tumor formation of MCF-7 tumor cells. Ex vivo transduction resulted in the inhibition of tumor formation and progression in the tumor xenograft model.

The primary modality for the treatment of cancer using gene therapy is the induction of apoptosis. This can be accomplished by either sensitizing the cancer cells to other agents or inducing apoptosis directly by stimulating intracellular pathways. Other cancer therapies take advantage of the need for the tumor to induce angiogenesis to supply the growing tumor with necessary nutrients. Endostatin and angiostatin are examples of two such therapies (WO 00/05356 and WO 00/26368).

In yet another embodiment, the treatment of a wide variety of cancerous states is within the scope of the invention. For example, melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, bronchial, lymphoma, brain, colon or bladder. In still more preferred embodiments said angiogenesis-related diseases is rheumatoid arthritis, inflammatory bowel disease, osteoarthritis, leiomyomas, ademonas, lipomas, hemangiomas, fibromas, vascular occlusion, restenosis, atherosclerosis, pre-neoplastic lesions, carcinoma in situ, oral hairy leukoplakia or psoriasis may be the subject of treatment. In particular embodiments, the cancer involves a tumor, which may or may not be resectable. Moreover, the cancer may involve metastatic tumor(s) or a tumor possibly capable of metastasis.

Cancer cells that may be treated by methods and compositions of the invention also include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments of the present invention, MDA-7 is provided as a nucleic acid expressing the MDA-7 polypeptide. In specific embodiments, the nucleic acid is a viral vector, wherein the viral vector dose is or is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles (vp). In certain embodiments, the viral vector is an adenoviral vector, a retroviral vector, a vaccinia viral vector, an adeno-associated viral vector, a polyoma viral vector, an alphaviral vector, a rhabdoviral vector, or a herpesviral vector. Most preferably, the viral vector is an adenoviral vector. In other specific embodiments, the nucleic acid is a non-viral vector.

In certain embodiments, the nucleic acid expressing the polypeptide is operably linked to a promoter. Non-limiting examples of promoters suitable for the present invention include a CMV IE, dectin-1, dectin-2, human CD11c, F4/80, SM22 or MHC class II promoter, however, any other promoter that is useful to drive expression of the mda-7 gene or the immunogene of the present invention, such as those set forth herein, is believed to be applicable to the practice of the present invention.

Preferably, the nucleic acid of the present invention is administered by injection. Other embodiments include the administering of the nucleic acid by multiple injections. In certain embodiments, the injection is performed local, regional or distal to a disease or tumor site. In some embodiments, the administering of nucleic acid is via continuous infusion, intratumoral injection, intraperitoneal, or intravenous injection. In other embodiments, the nucleic acid is administered to the tumor bed prior to or after; or both prior to and after resection of the tumor. Alternatively, the nucleic acid is administered to the patient before, during, or after chemotherapy, biotherapy, immunotherapy, surgery or radiotherapy. Preferably the patient is a human. In other embodiments the patient is a cancer patient.

II. MDA-7 Compositions

Examples of EGFR inhibitors are those agents that inhibit specifically EGFR and reduce or inhibit its activity. In particular embodiments, the EGFR inhibitor specifically inhibits the tyrosine kinase activity of EGFR. These EGFR tyrosine kinase inhibitor agents include, but are not limited to, AG1478, erlotinib (brand name Tarceva®), gefitinib (Iressa®), lapatinib (Tykerb®), and molecules derived from or related to AG1478. It is specifically contemplated that any agent listed above and in the table may also be disclaimed as part of the invention. In certain embodiments, the EGFR-targeted agent in the invention is AG1478 or a derivative thereof, which would or could include an of the PD compounds discussed above.

TABLE 1

| Brand Name | Generic or other name |
|---|---|
| Tarceva ® | Erlotinib |
| Iressa ® | Gefitinib |
| Tykerb | Lapatinib |
| | Canertinib |
| | Tyrphostin AG 825 |
| Erbitux ® | Cetuximab |
| | ABX-EGF (panitumumab) |
| | hR3 |
| | tyrphostin AG1318 |
| | tyrphostin RG13022 |
| | Tyrphostin |
| | Erbstatin |
| | RF14921 |
| | tyrphostin T23 |
| | tyrphostin T47 |
| | tyrphostin RG-13022 |
| | RG14620 |
| | tyrphostin AG879 |
| Matuzumab | EMD 72000 |

The tumor suppressor protein p53 has been implemented in a combination therapy with an EGFR inhibitor for the treatment of cancer. See U.S. patent application Ser. No. 11/150,521 filed on Jun. 10, 2005, which is hereby incorporated by reference in its entirety.

III. Nucleic Acids, Vectors and Regulatory Signals

The present invention concerns polynucleotides or nucleic acid molecules relating to the mda-7 gene and its gene product MDA-7. These polynucleotides or nucleic acid molecules are isolatable and purifiable from mammalian cells. It is contemplated that an isolated and purified MDA-7 encoding nucleic acid molecule, encoding either the secreted or full-length version, may take the form of RNA or DNA. As used herein, the term “RNA transcript” refers to an RNA molecule that is the product of transcription from a DNA nucleic acid molecule. Such a transcript may encode for one or more polypeptides.

As used in this application, the term “polynucleotide” refers to a nucleic acid molecule, RNA or DNA, that has been isolated free of total genomic nucleic acid. Therefore, a “polynucleotide encoding MDA-7” refers to a nucleic acid segment that contains MDA-7 coding sequences, yet is isolated away from, or purified and free of, total genomic DNA and proteins. When the present application refers to the function or activity of a MDA-7-encoding polynucleotide or nucleic acid, it is meant that the polynucleotide encodes a molecule that has the ability to induce apoptosis of a cancer cell.

The term “cDNA” is intended to refer to DNA prepared using RNA as a template. The advantage of using a cDNA, as opposed to genomic DNA or an RNA transcript is stability and the ability to manipulate the sequence using recombinant DNA technology (See Sambrook, 2001; Ausubel, 1996). There may be times when the full or partial genomic sequence is some. Alternatively, cDNAs may be advantageous because it represents coding regions of a polypeptide and eliminates introns and other regulatory regions.

It also is contemplated that a given MDA-7-encoding nucleic acid or mda-7 gene from a given cell may be represented by natural variants or strains that have slightly different nucleic acid sequences but, nonetheless, encode an MDA-7 polypeptide. In particular cases, a human MDA-7 polypeptide is a specific embodiment. Consequently, the present invention also encompasses derivatives of MDA-7 with minimal amino acid changes, but that possess the same activity.

The term “gene” is used for simplicity to refer to a functional protein, polypeptide, or peptide-encoding nucleic acid unit. As will be understood by those in the art, this functional term includes genomic sequences, cDNA sequences, and smaller engineered gene segments that express, or may be adapted to express, proteins, polypeptides, domains, peptides, fusion proteins, and mutants. The nucleic acid molecule encoding MDA-7 may comprise a contiguous nucleic acid sequence of the following lengths or at least the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128,129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more nucleotides, nucleosides, or base pairs. Such sequences may be identical or complementary to SEQ ID NO: 1 (human MDA-7 encoding sequence).

“Isolated substantially away from other coding sequences” means that the gene of interest forms part of the coding region of the nucleic acid segment, and that the segment does not contain large portions of naturally-occurring coding nucleic acid, such as large chromosomal fragments or other functional genes or cDNA coding regions. Of course, this refers to the nucleic acid segment as originally isolated, and does not exclude genes or coding regions later added to the segment by human manipulation.

In particular embodiments, the invention concerns isolated DNA segments and recombinant vectors incorporating DNA sequences that encode a MDA-7 protein, polypeptide or peptide that includes within its amino acid sequence a contiguous amino acid sequence in accordance with, or essentially as set forth in, SEQ ID NO:2, corresponding to the MDA-7 designated “human MDA-7” or “MDA-7 polypeptide.”

The term “a sequence essentially as set forth in SEQ ID NO:2” means that the sequence substantially corresponds to a portion of SEQ ID NO:2 and has relatively few amino acids that are not identical to, or a biologically functional equivalent of, the amino acids of SEQ ID NO:2.

The term “biologically functional equivalent” is well understood in the art and is further defined in detail herein. Accordingly, sequences that have or have at least or at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, and any range derivable therein, such as, for example, 70% to 80%, or 81% to 90%; or 91% to 99%; of amino acids that are identical or functionally equivalent to the amino acids of SEQ ID NO:2 will be sequences that are “essentially as set forth in SEQ ID NO:2” provided the biological activity of the protein is maintained with respect to inducing apoptosis. In particular embodiments, the biological activity of a MDA-7 protein, polypeptide or peptide, or a biologically functional equivalent, comprises enhancing an immune response. In certain other embodiments, the invention concerns isolated DNA segments and recombinant vectors that include within their sequence a nucleic acid sequence essentially as set forth in SEQ ID NO:1. The term “essentially as set forth in SEQ ID NO:1” is used in the same sense as described above and means that the nucleic acid sequence substantially corresponds to a portion of SEQ ID NO:1 and has relatively few codons that are not identical, or functionally equivalent, to the codons of SEQ ID NO:2. Again, DNA segments that encode proteins, polypeptide or peptides exhibiting MDA-7 activity will be employed in embodiments of the invention.

In particular embodiments, the invention concerns isolated nucleic acid segments and recombinant vectors incorporating DNA sequences that encode MDA-7 polypeptides or peptides that include within its amino acid sequence a contiguous amino acid sequence in accordance with, or essentially corresponding to MDA-7 polypeptides.

In some embodiments, a nucleic acid may encode an antisense construct. Antisense methodology takes advantage of the fact that nucleic acids tend to pair with “complementary sequences.” By complementary, it is meant that polynucleotides are those which are capable of base-pairing according to the standard Watson-Crick complementarity rules. Inclusion of less common bases such as inosine, 5-methylcytosine, 6-methyladenine, hypoxanthine and others in hybridizing sequences does not interfere with pairing.

Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

In certain embodiments, the nucleic acid encodes an interfering RNA. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery, 1999; Montgomery et al., 1998; Sharp and Zamore, 2000; Tabara et al., 1999). Activation of these mechanisms targets mature, dsRNA-complementary mRNA for destruction. Advantages of RNAi include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs are designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; or through an in vitro system derived from S2 cells. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

PCT publications WO 99/32619 and WO 01/68836 suggest that RNA for use in siRNA may be chemically or enzymatically synthesized. Both of these texts are incorporated herein in their entirety by reference. The contemplated constructs provide templates that produce RNAs that contain nucleotide sequences identical to a portion of the target gene. Typically the length of identical sequences provided is at least 25 bases, and may be as many as 400 or more bases in length. Longer dsRNAs may be digested to 21-25mer lengths with endogenous nuclease complex that converts long dsRNAs to siRNAs in vivo. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. U.S. Pat. No. 5,795,715 reports the simultaneous transcription of two complementary DNA sequence strands in a single reaction mixture, wherein the two transcripts are immediately hybridized.

Vectors of the present invention are designed, primarily, to transform cells with a therapeutic mda-7 gene or MDA-7 encoding nucleic acid sequence under the control of a eukaryotic promoter (i.e., constitutive, inducible, repressable, tissue specific). Also, the vectors may contain a selectable marker if, for no other reason, to facilitate their manipulation in vitro. However, selectable markers may play an important role in producing recombinant cells.

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term “promoter” will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In some embodiments, the promoter for use in the present invention is the cytomegalovirus (CMV) immediate early (IE) promoter. This promoter is commercially available from Invitrogen in the vector pcDNAIII, which is some for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

Another signal that may prove useful is a polyadenylation signal. Such signals may be obtained from the human growth hormone (hGH) gene, the bovine growth hormone (BGH) gene, or SV40.

The use of internal ribosome binding sites (IRES) elements are used to create multigene, or polycistronic, messages. IRES elements are able to bypass the ribosome scanning model of 5-methylatd cap-dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements which when positioned functionally upstream of a gene leads to the expression of that gene. Most transgene constructs of the present invention are functionally positioned downstream of a promoter element.

Compositions and methods of the invention are provided for administering the compositions of the invention to a patient.

A. Vectors

An MDA-7 polypeptide may be encoded by a nucleic acid molecule comprised in a vector. In this manner, an MDA-7 polypeptide can be provided to a patient through the administration of such a vector.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (2001) and Ausubel et al., 1996, both incorporated herein by reference. In addition to encoding a modified polypeptide such as modified gelonin, a vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Viral Vectors

a. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the some starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells.

b. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

c. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al., 1986; Lebkowski et al., 1988; McLaughlin et al., 1988), which means it is applicable for use with the present invention. Details concerning the generation and use of rAAV vectors are described in U.S. Pat. Nos. 5,139,941 and 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Ohi et al., 1990; Walsh et al., 1994; Wei et al., 1994). Recently, an AAV vector has been approved for phase I human trials for the treatment of cystic fibrosis.

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Protamine

Protamine may also be used to form a complex with an expression construct. Such complexes may then be formulated with the lipid compositions described above for adminstration to a cell. Protamines are small highly basic nucleoproteins associated with DNA. Their use in the delivery of nucleic acids is described in U.S. Pat. No. 5,187,260, which is incorporated by reference. U.S. patent application Ser. No. 10/391,068 (filed Mar. 24, 2003), which pertains to methods and compositions for increasing transduction efficiency of a viral vector by complexing the viral vector with a protamine molecule, is specifically incorporated by reference herein.

2. Non-Viral Delivery

In addition to viral delivery of the nucleic acid encoding a MDA-7 protein, the following are additional methods of recombinant gene delivery to a given host cell and are thus considered in the present invention.

a. Lipid Mediated Transformation

In a further embodiment of the invention, an expression vector may be entrapped in a liposome or lipid formulation. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Recent advances in lipid formulations have improved the efficiency of gene transfer in vivo (Smyth-Templeton et al., 1997; WO 98/07408). A novel lipid formulation composed of an equimolar ratio of 1,2-bis(oleoyloxy)-3-(trimethyl ammonio)propane (DOTAP) and cholesterol significantly enhances systemic in vivo gene transfer, approximately 150-fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these lipid structures include a positive colloidal stabilization by cholesterol, two dimensional DNA packing and increased serum stability.

Manufacture and use of such a formulation for the treatment of cancer is provided in Ser. No. 09/575,473, which is hereby incorporated by reference.

In further embodiments, the liposome is further defined as a nanoparticle. A “nanoparticle” is defined herein to refer to a submicron particle. The submicron particle can be of any size. For example, the nanoparticle may have a diameter of from about 0.1, 1, 10, 100, 300, 500, 700, 1000 nanometers or greater. The nanoparticles that are administered to a subject may be of more than one size.

Any method known to those of ordinary skill in the art can be used to produce nanoparticles. In some embodiments, the nanoparticles are extruded during the production process. Exemplary information pertaining to the production of nanoparticles can be found in U.S. Patent App. Pub. No. 20050143336, U.S. Patent App. Pub. No. 20030223938, U.S. Patent App. Pub. No. 20030147966, and U.S. Ser. No. 60/661,680, each of which is herein specifically incorporated by reference into this section.

In certain embodiments, an anti-inflammatory agent is administered with the lipid to prevent or reduce inflammation secondary to administration of a lipid:nucleic acid complex. For example, the anti-inflammatory agent may be a non-steroidal anti-inflammatory agent, a salicylate, an anti-rheumatic agent, a steroid, or an immunosuppressive agent. Information pertaining to administration of anti-inflammatory agents in conjunction with lipid-nucleic acid complexes can be found in U.S. Patent App. Pub. No. 20050143336, which is herein specifically incorporated by reference.

Synthesis of DOTAP:Chol nanoparticles is by any method known to those of ordinary skill in the art. For example, the method can be in accordance with that set forth in Chada et al., 2003, or Templeton et al., 1997, both of which are herein specifically incorporated by reference. DOTAP:Chol-DNA complexes were prepared fresh two to three hours prior to injection in mice.

One of ordinary skill in the art would be familiar with use of liposomes or lipid formulation to entrap nucleic acid sequences. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL).

Lipid-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of lipid-mediated delivery and expression of foreign DNA in cultured chick embryo, HeLa and hepatoma cells.

Lipid based non-viral formulations provide an alternative to adenoviral gene therapies. Although many cell culture studies have documented lipid based non-viral gene transfer, systemic gene delivery via lipid based formulations has been limited. A major limitation of non-viral lipid based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use subcutaneous, intradermal, intratumoral, or intracranial injection to avoid the toxicity and stability problems associated with cationic lipids in the circulation. The interaction of liposomes and plasma proteins is responsible for the disparity between the efficiency of in vitro (Felgner et al., 1987) and in vivo gene transfer (Zhu et al., 1993; Solodin et al., 1995; Liu et al., 1995; Thierry et al., 1995; Tsukamoto et al., 1995; Aksentijevich et al., 1996).

Recent advances in liposome formulations have improved the efficiency of gene transfer in vivo (WO 98/07408). A novel liposomal formulation composed of an equimolar ratio of 1,2-bis(oleoyloxy)-3-(trimethyl ammonio)propane (DOTAP) and cholesterol significantly enhances systemic in vivo gene transfer, approximately 150 fold. The DOTAP:cholesterol lipid formulation is said to form a unique structure termed a “sandwich liposome”. This formulation is reported to “sandwich” DNA between an invaginated bi-layer or ‘vase’ structure. Beneficial characteristics of these liposomes include colloidal stabilization by cholesterol, two dimensional DNA packing and increased serum stability. The particular use of a lipid nanoparticle with MDA-7 in the treatment of cancer is discussed in Ramesh et al., 2004, which is hereby incorporated by reference for the prepartion of the nanoparticle and its use.

The production of lipid formulations often is accomplished by sonication or serial extrusion of liposomal mixtures after (I) reverse phase evaporation (II) dehydration-rehydration (III) detergent dialysis and (IV) thin film hydration. Once manufactured, lipid structures can be used to encapsulate compounds that are toxic (chemotherapeutics) or labile (nucleic acids) when in circulation. Liposomal encapsulation has resulted in a lower toxicity and a longer serum half-life for such compounds (Gabizon et al., 1990). Numerous disease treatments are using lipid based gene transfer strategies to enhance conventional or establish novel therapies, in particular therapies for treating hyperproliferative diseases.

The liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

A nucleic acid for nonviral delivery may be purified on polyacrylamide gels, cesium chloride centrifugation gradients, column chromatography or by any other means known to one of ordinary skill in the art (see for example, Sambrook et al., 2001, incorporated herein by reference). In certain aspects, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, bulk of cellular components or in vitro reaction components, and/or the bulk of the total genomic and transcribed nucleic acids of one or more cells. Methods for isolating nucleic acids (e.g., equilibrium density centrifugation, electrophoretic separation, column chromatography) are well known to those of skill in the art.

IV. Proteins, Peptides and Polypeptides

The present invention is directed to methods and compositions of MDA-7 polypeptides. In certain embodiments, the MDA-polypeptides are used in the treatment of cancer. In certain embodiments, the MDA-7 polypeptide is directly provided. The terms “protein” and “polypeptide” are used interchangeably herein.

Additional embodiments of the invention encompass the use of a purified protein composition comprising MDA-7 protein and a truncated version of MDA-7 lacking its endogenous signal sequence or an MDA-7 polypeptide with a heterologous signal sequence. Truncated molecules of MDA-7 include, for example, molecules beginning approximately at MDA-7 amino acid residues 46-49 and further N-terminal truncations. Specifically contemplated are molecules starting at residue 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, and 182, and terminate at residue 206, and any contiguous length of amino acids therein. In additional embodiments, residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, and 48 are included with other contiguous residues of MDA-7, as shown in SEQ ID NO:2. In particular embodiments, MDA-7 is provided to a cell or subject as a full-length MDA-7 protein or a nucleic acid encoding a full-length MDA.7 protein, while in other embodiments, the secreted form of MDA-7 is provided as a protein or as a nucleic acid encoding the secreted MDA-7.

The present invention is also directed to methods and compositions of MDA-7 or nucleic acids encoding MDA-7 in combination with one or more EGFR inhibitors. In certain embodiments of the present invention, the EGFR inhibitor is a protein, polypeptide, or peptide; in particular embodiments, the EGFR inhibitor is protein or polypeptide that is an antibody.

As will be understood by those of skill in the art, modification and changes may be made in the structure of a MDA-7 polypeptide or peptide or EGFR inhibitor polypeptide or peptide, and still produce a molecule having like or otherwise desirable characteristics. For example, certain amino acids may be substituted for other amino acids or include deletions, additions, or truncations in the protein sequence without appreciable loss of interactive binding capacity with structures. Since it is the interactive capacity and nature of a protein that defines that protein's biological functional activity, certain amino acid sequence substitutions can be made in a protein sequence (or, of course, its underlying DNA coding sequence) and nevertheless obtain a protein with similar tumor suppressive, apoptosis-inducing, antiogenic, or cytokine properties. It is thus contemplated by the inventors that various changes may be made in the sequence of MDA-7 polypeptides or peptides (or underlying DNA) without appreciable loss of their biological utility or activity.

In terms of functional equivalents, the skilled artisan also understands it is also well understood by the skilled artisan that inherent in the definition of a biologically-functional equivalent protein or peptide, is the concept of a limit to the number of changes that may be made within a defined portion of a molecule that still result in a molecule with an acceptable level of equivalent biological activity. Biologically-functional equivalent peptides are thus defined herein as those peptides in which certain, not most or all, of the amino acids may be substituted. In particular, where small peptides are concerned, less amino acids may be changed. Of course, a plurality of distinct proteins/peptides with different substitutions may easily be made and used in accordance with the invention.

It is also well understood that where certain residues are shown to be particularly important to the biological or structural properties of a protein or peptide, e.g., residues in the binding site of an antibody, or in the apoptosis inducing region, such residues may not generally be exchanged.

Amino acid substitutions are generally based on the relative similarity of the amino acid side-chain substituents, for example, their hydrophobicity, hydrophilicity, charge, size, and the like. An analysis of the size, shape, and type of the amino acid side-chain substituents reveals that arginine, lysine, and histidine are all positively charged residues; that alanine, glycine, and serine are all a similar size; and that phenylalanine, tryptophan, and tyrosine all have a generally similar shape. Therefore, based upon these considerations, the following subsets are defined herein as biologically functional equivalents: arginine, lysine, and histidine; alanine, glycine, and serine; and phenylalanine, tryptophan, and tyrosine.

To effect more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, some, and those within ±0.5 are even more particularly preferred.

It is also understood in the art that the substitution of like amino acids can be made effectively on the basis of hydrophilicity, particularly where the biological functional equivalent protein or peptide thereby created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within 12 is preferred,some, those which are within ±1 are particularly preferred,some, and those within ±0.5 are even more particularly preferred.some.

While discussion has focused on functionally equivalent polypeptides arising from amino acid changes, it will be appreciated that these changes may be effected by alteration of the encoding DNA, taking into consideration also that the genetic code is degenerate and that two or more codons may encode the same amino acid.

A. In Vitro Protein Production

In addition to the purification methods provided in the examples, general procedures for in vitro protein production are discussed. Following transduction with a viral vector according to some embodiments of the present invention, primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshney, 1992).

One embodiment of the foregoing involves the use of gene transfer to immortalize cells for the production and/or presentation of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Another embodiment of the present invention uses autologous B lymphocyte cell lines, which are transfected with a viral vector that expresses an immunogene product, and more specifically, an protein having immunogenic activity. Other examples of mammalian host cell lines include Vero and HeLa cells, other B- and T- cell lines, such as CEM, 721.221, H9, Jurkat, Raji, etc., as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection: for dhfr, which confers resistance to; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

B. ER-Targeting Sequences

The polypeptides of the present invention include one or more endoplasmic reticulum targeting sequences. The final location of a protein within a cell depends upon targeting sequences encoded within the sequence of a protein. In the simplest case, the lack of a signal directs proteins to the default pathway which is the cytoplasm. Proteins destined to be retained in the ER must have certain signal peptides to retain the protein in the ER. The polypeptides of the present invention may or may not include additional amino acid residues at the N-terminal or C-terminal.

The ER is a network of membrane-enclosed tubules and sacs (cisternae) that extends from the nuclear membrane throughout the cytoplasm. The secretory pathway of proteins is as follows: rough ER→Golgi→secretory vesicles→cell exterior.

For proteins to be secreted, the protein must generally travel from the ER to the Golgi. However, there are certain proteins that must be maintained within the ER, such as BiP, signal peptidase, protein disulfide isomerase. Specific localization signals target proteins to the ER.

Certain proteins are retained in the ER lumen as a result of the presence of the ER targeting sequence Lys-Asp-Glu-Leu (KDEL, in the single-letter code(SEQ ID NO:3)) at their carboxy terminus. If this sequence is not part of the protein, the protein is instead transported to the Golgi and secreted from the cell. The presence of the KDEL sequence or the KKXX sequence (SEQ ID NO:4) at the carboxy terminus (KKXX sequences) results in retention of proteins in the ER. The presence of these sequences results in binding of the protein to specific recycling receptors in the membranes of these compartments and are then selectively transported back to the ER.

Protein export from the ER occurs not only by bulk flow, but by a regulated pathway that specifically recognizes targeting signals that mediate selective transport of proteins to the Golgi apparatus. The presence of a 16- to 30-residue ER signal sequence directs the ribosome to the ER membrane and initiates transport of the protein across the ER membrane.

ER signal sequences are usually located at the N-terminus of the protein. These targeting sequences frequently contains one or more positively charged amino acids followed by a continuous stretch of 6-12 hydrophobic residues. Signal sequences are usually cleaved from the protein while it is still growing on the ribosome. The specific deletion of several of the hydrophobic amino acids from a signal sequence or a mutation of one of them to a charged amino acid results in failure of the protein to cross the ER membrane into the lumen. The addition of random N-terminal amino acid sequences will cause a cytosolic protein to be translocated to the ER lumen, indicating that the hydrophobic residues form a binding site that is critical for ER targeting.

The endoplasmic reticulum targeting sequence may include any number of amino acid residues, as long as these amino acid residues target the destination of the polypeptide to the endoplasmic reticulum. The polypeptides of the present invention may include a single ER targeting sequence, or more than one ER targeting sequence. Additional information pertaining to ER targeting signals can be found in Invitrogen Catalog Nos. V890-20, V891-20, V892-20, and V893-20, “pShooter Vector Manual I (pEF/myc vectors),” on the internet at invitrogen.com/content/sfs/manuals/ pshooter_pef_man.pdf, which is hereby incorporated by reference in its entirety. Reviews of signal sequence recognition and protein targeting to the ER can also be found in Walter and Johnson, 1994; Koch et al., 2003; and Kabat et al., 1987, which are also specifically incorporated by reference herein.

C. Protein Production and Purification

The present invention employs purified MDA-7 in some embodiments of the invention. The following methods and similar methods known to one of ordinary skill in the art can be used to practice the methods of purification of MDA-7 disclosed herein. Such methods are disclosed in Ser. No. 10/791,692, which is hereby incorporated by reference. Part of this disclosure is provided below (without figures).

1. Antibody Production

a. Antibodies that Bind MDA-7

Recombinant his-tagged MDA-7 protein was produced in E. coli and was purified on a nickel NTA agarose column. The material was bound to the nickel resin in a batch mode for 45 minutes and then poured into a column and the eluate was run through the column bed. The material was washed with 10 mM Tris pH 8.0 containing 0.5% chaps and finally eluted off of the column with 10 mM Tris pH 8.0 plus 400 mM imidazole. The eluted MDA-7 was dialyzed against 10 mM Tris pH 8.0. The final product was shown to be a single band with a molecular weight of approx. 23 kDa. The amino terminal protein sequence was shown to be correct and purity was estimated to be greater than 90%.

This material was injected into rabbits using the following protocol: 400 mg MDA-7 protein with IFA and 100 mg of MDP was injected subcutaneously, 3 weeks later 200 μg MDA-7 protein with IFA was injected and 3 weeks after that another 100 mg of MDA-7 protein was injected intravenously. The titer of antiserum was shown to be greater than 1/100,000 based on an ELISA assay. Animals were boosted as needed.

The MDA-7 protein was coupled via sulfhydryl linkage to a solid support resin. The resin and bound protein was thoroughly washed. This washed material was used to make an MDA-7 column for antibody purification. The rabbit polyclonal sera was diluted 1:1 with 20 mM Tris buffer pH 8.0 and filtered through a 0.2-micron filter before being pumped onto the MDA-7 column. The column was then washed with the same 20 mM Tris buffer pH 8.0 until the absorbance returned to baseline. The antibody was eluted off the column with 0.1 M acetic acid. The eluent containing the antibody was immediately adjusted back to pH 8.0. This affinity-purified antibody was then dialyzed against 10 mM Tris pH 8.0 and concentrated.

b. Antibodies that Bind EGFR

Some embodiments of the present invention pertain to methods and compositions involving MDA-7 in combination with an inhibitor of EGFR, wherein the inhibitor is an antibody that binds EGFR.

As used herein, the term “antibody” refers to any form of antibody or fragment thereof that exhibits the desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

Included within the definition of an antibody that binds EGFR is an EGFR antibody binding fragment. As used herein, the term “EGFR binding fragment” or “binding fragment thereof” encompasses a fragment or a derivative of an antibody that still substantially retain its biological activity of inhibiting EGFR activity. Therefore, the term “antibody fragment” or EGFR binding fragment refers to a portion of a full length antibody, generally the antigen binding or variable region thereof. Examples of antibody fragments include Fab, Fab′, F(ab′).sub.2, and Fv fragments; diabodies; linear antibodies; single-chain antibody molecules, e.g., sc-Fv; and multispecific antibodies formed from antibody fragments. Typically, a binding fragment or derivative retains at least 50% of its EGFR inhibitory activity. Preferably, a binding fragment or derivative retains about or at least about 60%, 70%, 80%, 90%, 95%, 99% or 100% of its EGFR inhibitory activity. It is also intended that a EGFR binding fragment can include conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Any suitable method for generating monoclonal antibodies may be used. For example, a recipient may be immunized with EGFR or a fragment thereof. Any suitable method of immunization can be used. Such methods can include adjuvants, other immunostimulants, repeated booster immunizations, and the use of one or more immunization routes.

Any suitable source of EGFR can be used as the immunogen for the generation of the non-human antibody of the compositions and methods disclosed herein. Such forms include, but are not limited whole protein, peptide(s), and epitopes, generated through recombinant, synthetic, chemical or enzymatic degradation means known in the art.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

2. Purification and Characterization of Secreted MDA-7 Using Polyclonal Antibodies

a. Affinity Column Production

Different polyclonal antibodies against human MDA-7 from rabbit serum were first purified. Frozen rabbit serum samples were thawed and diluted 1:1 with sterile 1×PBS buffer. The diluted samples were individually exposed in bath method at 4° C. overnight with gentle rocking to 2 mls Protein A-Sepharose (SIGMA). Four different columns were generated. The resin was washed with 10 column volumes of 20 mM sodium phosphate dibasic (61 mls) to make a pH of 7.0 The column was eluted with 3 column volumes of 0.15 M NaCl (pH 3.0) in three aliquots and neutralized with 0.5M HEPES. A Bradford Protein Assay (BioRad) was used to quantify the eluted antibody. The antibody was then exchanged into 0.1 M NaHCO₃ (pH 8.3) containing 0.5 M NaCl, by dialyzing overnight in a 10,000 MWCO dialysis cassette.

To activate the dried CNBr-Sepharose, 1 gram was washed with 10-15 column volumes with 1 mM cold HCl. Serial volumes of 5 mls were used to ensure removal of sucrose. Activated CNBr-Sepharose was then washed with 10 column volumes by serial washings of 1 column volume to exchange into 0.1 M NaHCO₃, pH 8.3. In each case, approximately 80-90 milligrams of antibody was recovered after purification and buffer exchange. Then 5 mls of swollen activated CNBr-Sepharose was incubated with 80-90 milligrams of purified antibody in 0.1 M NaHCO₃, pH 8.3, for 4 hours at room temperature with gentle rotation.

Antibody binding efficiency was determined by Bradford Protein assay, and in each case was greater than 95% of the antibody bound to the activated CNBr-Sepharose. After coupling, non-reacted groups were blocked by washing 25-30 column volumes in 0.1 M Tris, pH 8.0. The column was then washed with serial washes of 0.1 M Tris, pH 8.0, 0.5 M NaCl, 5×column volumes 5 times, alternating with 0.1 M acetate buffer, pH 4.0, 0.5 M NaCl. Protein estimation was performed on the washes and no protein was detected.

b. Affinity Chromatography Purification

Stably transfected 293 T cells that secrete soluble, glycosylated MDA-7 were obtained and maintained at high confluency in RPMI containing 5% Fetal Calf Serum with 1:100 L-glutamine, 1:100 pen/strep and 1:100 HEPES. Cells were split every two-three days with alternation every 7 days of maintenance in 1:1000 dilution hygromycin, (20 mg/ml stock). Then 400 mls of supernatant was harvested every 2-3 days and concentrated with an AMICON stirred cell over a 10,000 molecular weight cutoff membrane. 50 mls of concentrated supernatant was exposed in batch method to 5 mls bed volume of antibody-CNBr-sepharose, (affinity resin) for 2 days at 4° C. with gentle rocking. The affinity resin was then placed in a Pharmacia XK 26 column and the supernatant passed through three times to ensure maximum binding of antigen to antibody. The affinity resin was washed with 5×20 mls 0.1 M Tris pH 8.0 by gravity flow. MDA-7 was eluted with 3×5 mls 1 M NaCl, 0.1 M Glycine, pH 3.0 and immediately neutralized with 0.5 mls HEPES buffer. Immediately after elution and neutralization, 2 mgs of human albumin was added to protect against protein loss. The eluted protein was then concentrated over 10,000 molecular weight cutoff spin columns (AMICON), and exchanged into sterile 1× PBS. Then 1-1.5 mls of 1×PBS exchanged affinity purified protein was exposed to 200 microliters 3× washed Protein-A Sepharose (SIGMA) for 2 hours at room temperature with rotation, or over night at 4° C. with rotation. Protein A exposure absorbs antibody that leaches into the elution fraction.

Four different polyclonal antibodies, whose production is described herein, were tested in affinity purification. Size resolution purification (see Size Exclusion) was employed to removed significant contaminating protein from the supernatant prior to affinity purification, the most abundant of which was bovine serum albumin (BSA). However, exposure of MDA-7 isolated in this fashion failed to permit the antibody on the column to retain MDA-7. This was probably due to BSA blocking non-specific binding sites that could retain MDA-7 in the absence of BSA. MDA-7 is a highly glycosylated protein it is considered very capable of sticking to plastic and other surfaces.

Removal of BSA from MDA-7 containing supernatant inhibits purification of MDA-7 by affinity chromatography. Most protein was present in the flow through. No MDA-7 protein is retained on the affinity column until elution. Affinity purifications that contained significant amounts of BSA, (2-3 mgs/ml by silver stain) retained biological function for longer than the purifications wherein the BSA contamination was significantly less. Affinity purification in the presence of BSA permits the retention of MDA-7 on the affinity column until elution with high molar NaCl and low pH. Affinity purification by polyclonal affinity resin resulted in multiple lots with relatively similar amounts of MDA-7. Coomassie analysis indicated relatively low quantities of contaminating protein. Purification of MDA-7 of greater than about 20% homogeneity was observed.

Affinity purification was repeatable and enriched the MDA-7 to relative purity by coomassie stain analysis of 12% polyacrylamide gels. By intensity of bands detected on the Western blot, more MDA-7 was retained with longer exposure of the antigen to the affinity resin. There was little difference between the method of exchange into 1× PBS, when comparing the dialysis cassette and the spin columns.

c. Anion Exchange Purification

Two to three lots of affinity purified MDA-7 were pooled and exchanged into 50 mM MES, pH 5.0 in a 10,000 MWCO dialysis cassette from 2-12 hrs at room temperature. Protein was then loaded onto a 5 ml bed volume anion exchange column at a flow rate of 1 ml/minute. 10 mls of flow through were taken and the bound protein was eluted with a step gradient of 1 M NaCl in 50 mM MES, pH 5.0. The elution program began with a 10 ml wash of 50 mM MES, pH 5.0 at flow rate of 2 mls/min. The first step elution was from 0 M to 0.25 M NaCl in 5 minutes with a 5 minute wash at 50 mM MES, 0.25 M NaCl, pH 5.0. The second gradient step was from 0.25 M NaCl to 0.5 M NaCl in 5 minutes followed by a 5 minute wash. The final elution was from 0.5 M NaCl to 1 M NaCl. MDA-7 was retained on to column until elution with 0.9-1.0 M NaCl; MDA-7 was purified to about 90%-95% homogeneity.

The unglycosylated protein of 18 KDa did not bind to the anion exchange column at pH 5.0. Silver stain analysis of fractions from post-affinity anion exchange of MDA-7 revealed that the unglycosylated form of MDA-7 is not associated with the co-purifying glycosylated proteins. The native MDA-7 complex appears to contain at least three proteins of molecular weight 31, 28 and 27/26. Previously, an attempt was made to purify MDA-7 utilizing a one step anion exchange purification, wherein the supernatant containing MDA-7 was exchanged into 50 mM MES, pH 6.0. One step anion exchange purification demonstrated that each peak from the anion exchange column contains MDA-7 detected by polyclonal anti-MDA-7 on western blot. Purification by this method failed to significantly enrich for MDA-7 at any range of ionic strength, as MDA-7 leached from the column at all molarities of NaCl employed.

d. Size Exclusion Chromatography

A 200 ml bed volume size exclusion chromatography column was generated utilizing S200 Sephadex (Pharmacia) poured into an XK 26 1 meter column (Pharmacia). The column was allowed to gravity settle, and was then packed at 3.5 mls/min with a BioRad BioLogic Workstation.

To determine the apparent molecular weight of MDA-7 secreted by the 293 t cells, protein molecule weight standards, (mouse IgG 5 mgs, Alkaline Phosphatase 3 mgs, BSA 10 mgs, and human beta2microglobulin 3 mgs) were combined to determine the relative retention times. Elution times of the purified proteins relative to molecular weights were plotted and an R² value of 0.97 derived. 200 mls of 293 t supernatant containing MDA-7 was concentrated over a 10,000 MWCO filter in an AMICON stirred cell down to 10 mls and loaded at 2 mls/min in 1×PBS on the size resolution column. Fractions were taken every 5 mls. Relative retention times was determined by Western blot analysis of sequential samples and compared to the line derived from the known standards. An apparent molecular weight of 80-100 kDa was assigned to the associated MDA-7. Less than 0.1 % of the total MDA-7 present was found to be in monomeric 31 kDa form. MDA-7 complex was eluted at between a molecular weight of about 85-95 kDa.

e. Size, Anion, and Lectin Purification

Lectin purification over a ConcanavalinA-Sepharose column was employed in an attempt to purify MDA-7. However, no net increase in relative purity was achieved. Combinatorial purifications, wherein size exclusion, anion, and lectin purification methods, were utilized in all combinations to enrich for MDA-7. However, no combination of these methods provided for greater purification of MDA-7 than affinity chromatography followed by anion chromatography. These results demonstrate that MDA-7 can be purified to at least 90-95% homogeneity by affinity and anion exchange chromatography.

3. Purification and Characterization of Secreted MDA-7 Using Monoclonal Antibodies

a. Antibody Production

The hybridoma clone, designated 7G11F.2 (monoclonal antibody), was determined to produce antibody that was the most effective at detecting IL-24/mda-7 positive cells by intracellular FACS analysis of stably transfected 293t cells that had been treated with Brefeldin A. Based upon these preliminary data, this clone was utilized to produce 5 liters of supernatant. Briefly cells, (7G11F.2) were seeded at 1×10⁶ cells/ml in 50 mls of DMEM supplemented with containing 10% Fetal Calf Serum with 1:100 L-glutamine, 1:100 pen/strep and 1:100 HEPES. Cells were seeded and permitted to grow for 10 days, then the supernatant was harvested.

b. Antibody Purification

Supernatant was clarified of cells by centrifugation at 2000 rpm for 10 minutes and decanted. The clarified supernatant was then sterile filtered over a 0.22 micro cellulose acetate filter and concentrated with an Amicon Stirred Cell under nitrogen over a YMCO 30 kDa membrane to 50 mls. The concentrated supernatant was exposed to rProtein G cross-linked to sepharose, (Sigma) o/n at 4° C. The antibody was eluted with 1 M NaCl pH 3.0, 3 column volumes in three aliquots and neutralize with 0.5 M HEPES. To remove contaminating bovine IgG, the resulting eluate was exchanged into 1×PBS containing 0.4 M NaCl (total), via dialysis cassette (Pierce/Endogen, YMCO 30 kDa). The protein was exposed to rProtein A crosslinked to sepharose, (Sigma) o/n 4° C. The flow through from the column was taken, as the protein A binds the bovine IgG with higher affinity than the mouse IgG1a. Relative purity was determined by analysis on SDS PAGE and taken to be 90% pure, (7G11F.2) with the contaminating protein wholy comprised of bovine IgG. Bradford Protein Assay, (BioRad), was used to quantify eluted antibody. The antibody was then exchanged into 0.1 M NaHCO₃, pH 8.3 containing 0.5 M NaCl, by dialyzing overnight in a 10,000 MWCO dialysis cassette.

c. Affinity Column Production

To activate dried CNBr-Sepharose, 1 gram was washed with 10-15 column volumes of 1 mM cold HCl. Serial volumes of 5 mls were used to ensure removal of sucrose. Activated CNBr-Sepharose was then washed with 10 column volumes by serial washings of 1 column volume to exchange into 0.1 M NaHCO₃, pH 8.3. 25 mgs of antibody, (7G11F.2) was recovered after purification and buffer exchange. 2 mls of swollen, activated CNBr-Sepharose was incubated with the purified antibody in 0.1 M NaHCO3, pH 8.3 for 4 hours at room temperature with gentle rotation.

Antibody binding efficiency was determined by Bradford Protein Assay; greater than 95% of the antibody bound to the activated CNBr-Sepharose.

After coupling, non-reacted groups were blocked by washing 25-30 column volumes in 0.1 M Tris pH 8.0. Finally the column was washed with serial washes of 0.1 M Tris pH 8.0, 0.5 M NaCl, 5×column volume 5 times alternating with 0.1 M acetate buffer, pH 4.0, 0.5 M NaCl. Protein estimation was performed on the washes and no protein was detected.

d. Affinity Purification

Stably transfected 293t cells that secrete soluble, glycosylated IL-24 were obtained from Introgen, Inc. and maintained at high confluency in RPMI containing 5% Fetal Calf Serum with 1:100 L-glutamine, 1:100 pen/strep and 1:100 HEPES. Cells were split every two-three days with alternation every 7 days of maintenance in 1:1000 dilution hygromycine, (20 mg/ml stock). 400 mls of supernatant is harvested every 2-3 days and concentrated with an Amicon stirred cell over a 10,000 molecular weight cutoff membrane. 50 mls of concentrated supernatant is exposed in batch method to 5 mls bed volume of antibody-CNBr-sepharose, (affinity resin) for 2 days at 4° C. with gentle rocking. The affinity resin was placed in a Pharmacia XK 26 column and the supernatant passed through three times to ensure maximum binding of antigen to antibody. The affinity resin was washed with 5×20 mls 0.1 M Tris pH 8.0 by gravity flow. IL-24 was eluted with 3×5 mls 1 M NaCl, 0.1 M Glycine, pH 3.0 and immediately neutralized with 0.5 mls HEPES buffer. Immediately after elution and neutralization, 2 mgs of Human Albumin was added to protect against protein loss. The eluted protein was then concentrated over 10,000 molecular weight cutoff spin columns, (Amicon) and exchanged into sterile 1×PBS. 1-1.5 mls of 1×PBS exchanged affinity purified protein was exposed to 200 microliters 3× washed rProtein-A Sepharose, (Sigma) for 2 hours at room temperature with rotation, or overnight at 4° C. with rotation. Protein A exposure absorbed antibodies that leached into the elution and its removal is crucial for maintaining IL-24 function.

The 7G11F.2 monoclonal antibody column retained similar amounts of IL-24/mda-7 as the polyclonal columns in the previous section.

The following general techniques are also well known and can be used to implement purification methods.

4. Gel Electrophoresis

Gel electrophoresis is a well-known technique that can be used in the purification procedure. Agarose, agarose-acrylamide or polyacrylamide gel electrophoresis using standard methods (Sambrook et al., 2001) can be utilized in the purification process.

5. Chromatographic Techniques

Alternatively, chromatographic techniques may be employed to effect isolation and purification of MDA-7. There are many kinds of chromatography which may be used in the present invention: adsorption, affinity, partition, ion-exchange and molecular sieve, and many specialized techniques for using them including column, paper, thin-layer and gas chromatography (Freifelder, 1982).

6. Immunological Reagents

Certain aspects of the claimed invention involve use of immunological reagents. In certain embodiments of the claimed invention, immunological reagents are used in the purification of preparations of MDA-7. Antibodies are contemplated for use with purification methods. Such antibodies can be readily created and/or are readily available.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

However, “humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's dental disease are likewise known and such custom-tailored antibodies are also contemplated.

The methods for generating monoclonal antibodies (MAbs) is well known to those of skill in the art.

V. Pharmaceutical Formulations and Delivery

In certain embodiments of the present invention, methods involve providing an MDA-7 protein (full-length or secreted form) either as purified protein or as an expression construct encoding such an MDA-7 protein. Moreover, methods involve providing an EGFR inhibitor as well.

A. Effective Amount

An “effective amount” of the pharmaceutical composition, generally, is defined as that amount sufficient to detectably and repeatedly to achieve the stated desired result, for example, to ameliorate, reduce, minimize or limit the extent of the disease or its symptoms. More rigorous definitions may apply, including reduction in tumor growth rate, reduction in tumor size, inhibition of metastasis of primary tumor, inhibition of metastases (number or size, induction of apoptosis of cancer or tumor cells, sensitization to other cancer therapy such as radiotherapy or chemotherapy, prevention of recurrence, induction of remission, halting tumor growth, increased life span, or reduction in amount (courses and/or strength of doses) of other cancer therapy.

B. Administration

In certain specific embodiments, it is desired to kill cells, inhibit cell growth, inhibit metastasis, decrease tumor or tissue size and otherwise reverse or reduce the malignant phenotype of tumor cells, induce an immune response, or inhibit angiogenesis using the methods and compositions of the present invention. The routes of administration will vary, naturally, with the location and nature of the lesion or site to be targeted, and include, e.g., intradermal, intrathecal, intratumoral, subcutaneous, regional, parenteral, intravenous, intramuscular, intranasal, systemic, and oral administration and formulation.

Direct injection, intratumoral injection, or injection into the tumor vasculature is specifically contemplated for discrete, solid, accessible tumors or other accessible target areas. Local, regional or systemic administration also may be appropriate. For tumors of >4 cm, the volume to be administered will be about 4-10 ml (preferably 10 ml), while for tumors of <4 cm, a volume of about 1-3 ml will be used (preferably 3 ml).

Multiple injections delivered as single dose comprise about 0.1 to about 0.5 ml volumes. The viral particles may advantageously be contacted by administering multiple injections to the tumor or targeted site, spaced at approximately 1 cm intervals.

In the case of surgical intervention, the present invention may be used preoperatively, to render an inoperable tumor subject to resection. Alternatively, the present invention may be used at the time of surgery, and/or thereafter, to treat residual or metastatic disease. For example, a resected tumor bed may be injected or perfused with a formulation comprising MDA-7 or an MDA-7-encoding construct together with or in the absence of an immunogenic molecule. The perfusion may be continued post-resection, for example, by leaving a catheter implanted at the site of the surgery. Periodic post-surgical treatment also is envisioned.

Continuous perfusion of protein, an expression construct or a viral construct also is contemplated. The amount of construct or peptide delivered in continuous perfusion can be determined by the amount of uptake that is desirable.

Continuous administration also may be applied where appropriate, for example, where a tumor or other undesired affected area is excised and the tumor bed or targeted site is treated to eliminate residual, microscopic disease. Delivery via syringe or catherization is some. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

Treatment regimens may vary as well, and often depend on tumor type, tumor location, immune condition, target site, disease progression, and health and age of the patient. Obviously, certain types of tumors will require more aggressive treatment, while at the same time, certain patients cannot tolerate more taxing protocols. The clinician will be best suited to make such decisions based on the known efficacy and toxicity (if any) of the therapeutic formulations.

In certain embodiments, the tumor or affected area being treated may not, at least initially, be resectable. Treatments with therapeutic viral constructs may increase the resectability of the tumor due to shrinkage at the margins or by elimination of certain particularly invasive portions. Following treatments, resection may be possible. Additional treatments subsequent to resection will serve to eliminate microscopic residual disease at the tumor or targeted site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Unit dose of the present invention may conveniently be described in terms of plaque forming units (pfu) or viral particles for a viral construct. Unit doses range from 10³, 10⁴, 10⁵, 10⁶, 10⁷, 10⁸, 10⁹, 10¹⁰, 10¹¹, 10¹², 10¹³ pfu or viral particles (vp) and higher. Alternatively, the amount specified may be the amount administered as the average daily, average weekly, or average monthly dose.

Protein may be administered to a patient in doses of about or of at least about 0.01. 0.05, 0.1, 0.2, 0.3, 0.4, 0.5, 0.6, 0.7, 0.8, 0.9, 1.0, 2.0, 3.0, 4.0, 5.0, 6.0, 7.0, 8.0. 9.0, 10, 15, 20, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 or more ng/ml, mg/kg (kg is weight of subject), or mg/mm² (mm² is two dimensional size of tumor) or any range derivable therein. Alternatively, any amount specified herein may be the amount administered as the average daily, average weekly, or average monthly dose.

EGFR inhibitors can be administered to the patient in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 mg, μmol, or mmol, or any range derivable therein. Alternatively, the amount specified may be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area).

In certain embodiments, the EGFR inhibitor is erlotinib, which can be administered or ingested in doses of about, at least about, or at most about, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 mg, or any range derivable therein. In specific embodiments, the dose is about 25, 100, or 150 mg. In further embodiments, Erlotinib is orally ingested by the patient in the form of a tablet or pill.

c. Injectable Compositions and Formulations

In some embodiments, the method for the delivery of an immunogenic molecule, an expression construct encoding a MDA-7 protein, MDA-7 protein, and/or an an EGFR inhibitor is via systemic administration. However, the pharmaceutical compositions disclosed herein may alternatively be administered parenterally, subcutaneously, directly, intratracheally, intravenously, intradermally, intramuscularly, or even intraperitoneally as described in U.S. Pat. Nos. 5,543,158; 5,641,515 and 5,399,363 (each specifically incorporated herein by reference in its entirety).

Injection of nucleic acid constructs may be delivered by syringe or any other method used for injection of a solution, as long as the expression construct can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain formulations, a water-based formulation is employed while in others, it may be lipid-based. In particular embodiments of the invention, a composition comprising an MDA-7 (or encoding nucleic acid) and/or an EGFR inhibitor is in a water-based formulation. In other embodiments, the formulation is lipid based.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the some methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

Compounds and agents may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The MDA-7 protein (or fragments thereof) or a nucleic acid encoding all or part of MDA-7, as well as an EGFR inhibitor, may be formulated as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In certain formulations, an EGFR inhibitor is formulated as a dry power. It is a further object of the present invention to use, for the process, readily accessible cheap raw materials in the form of dairy by-products, in place of pure carbohydrates.

The compounds are administered in a manner compatible with the dosage formulation, and in such amount as will be therapeutically effective. The quantity to be administered depends on the subject to be treated, including, e.g., the aggressiveness of the cancer, the size of any tumor(s), the previous or other courses of treatment. Precise amounts of active ingredient required to be administered depend on the judgment of the practitioner. Suitable regimes for initial administration and subsequent administration are also variable, but are typified by an initial administration followed by other administrations. Such administration may be systemic, as a single dose, continuous over a period of time spanning 10, 20, 30, 40, 50, 60 minutes, and/or 1, 2, 3, 4, 5, 6, 7, 8, 9, 10, 11, 12, 13, 14, 15, 16, 17, 18, 19, 20, 21, 22, 23, 24 or more hours, and/or 1, 2, 3, 4, 5, 6, 7, days or more. Moreover, administration may be through a time release or sustained release mechanism, implemented by formulation and/or mode of administration.

The manner of application may be varied widely. Any of the conventional methods for administration are applicable. These are believed to include oral application on a solid physiologically acceptable base or in a physiologically acceptable dispersion, parenterally, by injection or the like. The dosage will depend on the route of administration and will vary according to the size of the host. In many instances, it will be desirable to have multiple administrations of each of both of the therapeutic agents (MDA-7 and EGFR inhibitor).

D. Combination Treatments

In certain embodiments, the compositions and methods of the present invention involve an MDA-7 polypeptide, or expression construct coding therefor, and an EGFR inhibitor to enhance the effect of MDA-7 or to increase any therapeutic, diagnostic, or prognostic effect for which the MDA-7 and/or EGFR inhibitor is being employed. These compositions would be provided in a combined amount effective to achieve the desired effect, for example, the killing of a cancer cell and/or the inhibition of angiogenesis. This process may involve contacting the cells with the expression construct and the agent(s) or multiple factor(s) at the same time. This may be achieved by contacting the cell with a single composition or pharmacological formulation that includes both or all agents, or by contacting the cell with two or more distinct compositions or formulations, at the same time, wherein one composition provides 1) MDA-7 (either as a protein or nucleic acid); and/or 2) the EGFR inhibitor; and/or 3) the third agent(s).

In embodiments of the present invention, it is contemplated that an mda-7 gene (or cDNA) or protein therapy is used in conjunction with an EGFR inhibitor (referred to as “MDA-7/EGFR inhibitor therapy”), in addition to a second or other anti-cancer agents or therapies. Alternatively, the MDA-7/EGFR inhibitor therapy therapy may precede or follow the other anti-cancer treatment by intervals ranging from minutes to weeks. In embodiments where the MDA gene or protein therapy is provided to the patient separately from the EGFR inhibitor, one would generally ensure that a significant period of time did not expire between the time of each delivery, such that the two compounds would still be able to exert an advantageously combined effect on the patient; alternatively, in embodiments where the MDA-7/EGFR inhibitor therapy is provided to the patient separately from the second anti-cancer therapy, one would generally ensure that a significant period of time did not expire between the time of each therapy, such that the two therapies would still be able to exert an advantageously combined effect on the patient. In such instances, it is contemplated that one may provide a patient with either 1) the MDA-7/EGFR inhibitor therapy and the second anti-cancer therapy within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed, for example MDA gene or protein therapy is “A” and the EGFR inhibitor is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Alternatively, “A” could be an administration of the MDA-7/EGFR inhibitor therapy and “B” the administration of a second anti-cancer therapy. In other embodiments, MDA-7 gene or protein therapy is “A” and the EGFR inhibitor is “B”; or, “B” could be an administration of the MDA-7/EGFR inhibitor therapy and “A” the administration of a second anti-cancer therapy.

Administration of any compound or therapy of the present invention to a patient will follow general protocols for the administration of such compounds, taking into account the toxicity, if any, of the vector or any protein or other agent. Therefore, in some embodiments there is a step of monitoring toxicity that is attributable to MDA-7 and/or an EGFR inhibitor. It is expected that the treatment cycles would be repeated as necessary. It also is contemplated that various standard therapies, as well as surgical intervention, may be applied in combination with the described therapy.

In specific embodiments, it is contemplated that a second anti-cancer therapy, such as chemotherapy, radiotherapy, immunotherapy or other gene therapy, is employed in combination with the MDA-7/ EGFR inhibitor therapy.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

c. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with MDA-7/EGFR inhibitor therapy. The general approach for combined therapy is discussed below. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor such as MDA-7 has been shown to enhance anti-tumor effects (Ju et al., 2000). Moreover, antibodies against any of these compounds can be used to target the anti-cancer agents discussed herein.

As discussed earlier, examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005; 5,739,169; Hui and Hashimoto, 1998; Christodoulides et al., 1998), cytokine therapy e.g., interferons α, β and γ; IL-1, GM-CSF and TNF (Bukowski et al., 1998; Davidson et al., 1998; Hellstrand et al., 1998) gene therapy e.g., TNF, IL-1, IL-2, p53 (Qin et al., 1998; Austin-Ward and Villaseca, 1998; U.S. Pat. Nos. 5,830,880 and 5,846,945) and monoclonal antibodies e.g., anti-ganglioside GM2, anti-HER-2, anti-p185; Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Table 2 contains a non-limiting list of several known anti-cancer immunotherapeutic agents and their targets. It is contemplated that one or more anti-cancer therapies may be employed with the MDA-7 therapies described herein.

TABLE 2

| Generic Name | Target |
|---|---|
| cetuximab | EGFR |
| panitumumab | EGFR |
| trastuzumab | erbB2 receptor |
| bevacizumab | VEGF |
| alemtuzumab | CD52 |
| gemtuzumab ozogamicin | CD33 |
| rituximab | CD20 |
| tositumomab | CD20 |
| matuzumab | EGFR |
| ibritumomab tiuxetan | CD20 |
| tositumomab | CD20 |
| HuPAM4 | MUC1 |
| MORAb-009 | mesothelin |
| G250 | carbonic anhydrase IX |
| mAb 8H9 | 8H9 antigen |
| M195 | CD33 |
| ipilimumab | CTLA4 |
| HuLuc63 | CS1 |
| alemtuzumab | CD53 |
| epratuzumab | CD22 |
| BC8 | CD45 |
| HuJ591 | Prostate specific membrane antigen |
| hA20 | CD20 |
| lexatumumab | TRAIL receptor-2 |
| pertuzumab | HER-2 receptor |
| Mik-beta-1 | IL-2R |
| RAV12 | RAAG12 |
| SGN-30 | CD30 |
| AME-133v | CD20 |
| HeFi-1 | CD30 |
| BMS-663513 | CD137 |
| volociximab | anti-α5β1 integrin |
| GC1008 | TGFβ |
| HCD122 | CD40 |
| siplizumab | CD2 |
| MORAb-003 | folate receptor alpha |
| CNTO 328 | IL-6 |
| MDX-060 | CD30 |
| ofatumumab | CD20 |
| SGN-33 | CD33 |

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient (Irie and Morton, 1986; Irie et al., 1989; Bajorin et al., 1988).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and re-administered (Rosenberg et al., 1988;. 1989).

d. Gene Therapy

In yet another embodiment, a combination treatment involves gene therapy in which a therapeutic polynucleotide is administered before, after, or at the same time as an MDA-7 polypeptide or nucleic acid encoding the polypeptide. Delivery of an MDA-7 polypptide or encoding nucleic acid in conjunction with a vector encoding another gene products may have a combined therapeutic effect on target tissues.

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs'surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 1 1, or 12 months. These treatments may be of varying dosages as well.

f. Hormonal Therapy

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synergistic Anti-Tumor Effect with Erlotinib and AD-MDA7

A. Materials and Methods

Cell Lines—All cell lines were obtained from American Type Culture Collection (ATCC, Rockville, Md.). The non small cell lung cancer cell line H1299 was cultured as previously described (Saeki et al., 2000). The prostate cancer cell line LNCaP was cultured as recommended by ATCC. Briefly, all cells were maintained in RPMI 1640 supplemented with 10% fetal bovine serum, 10 mM glutamine, 100 units/ml penicillin, 100 mg/ml streptomycin (Life Technologies, Inc., Grand Island, N.Y.) in a 5% CO₂ atmosphere at 37° C.

Recombinant Adenovirus—The recombinant adenovirus vectors carrying the mda-7 gene (Ad-mda7) and the luciferase reporter gene (Ad-luc) were obtained from Introgen Therapeutics (Introgen Therapeutics, Houston, Tex.). Production of the replication-deficient human type 5 Adenovirus (Ad5) containing the mda-7 gene (Ad-mda7) or luciferase reporter gene (Ad-luc) have been previously reported (Saeki et al., 2000; Mhashilkar, 2001; Pataer et al., 2002). Construction of Ad-mda7 involved linking mda-7 cDNA to a CMV-IE promoter, followed by an SV40 polyadenylation [p(A)] sequence; this expression cassette was placed in the E1 region of Ad5. The same adenoviral vector containing the sequence for expression of the green fluorescent protein (Ad-GFP) was used as control virus.

Cell Viability Assay—The effect of erlotinib and Ad-mda7 on human non-small cell lung cancer (H1299) and human prostate cancer (LNCaP) cell lines was evaluated by cell counting after trypan blue (Invitrogen Co., Carlsbad, Calif.) exclusion assay. Briefly, H1299 and LNCaP cell lines (5×10⁴ cells/well in 6-well plates) were treated with PBS or transduced with either Ad-luc or Ad-mda7 at an MOI of 2000 viral particles (vp)/cell. Erlotinib, dissolved in DMSO, was administered to both transduced and PBS treated cell cultures at a dose of either 2.5 or 5 μM per well. Cells not administered erlotinib (either PBS treated or transduced) served as controls. Seventy-two hours after adenoviral vector transduction, cells were trypsinized and an aliquot suspended 1:1 volume with 0.4% trypan blue. Total cell numbers and cell viability counts were assessed using a hemocytometer by light microscopy (Chada et al., 2005).

Transduction Efficiency Assay—The effect of erlotinib on adenoviral vector transduction efficiency of H1299 and LNCaP cell lines was evaluated. Briefly, H1299 and LNCaP cell lines (5×10⁴ cells/well in 6-well plates) were transduced with Ad-GFP at 50 or 100 vp/cell in the presence of either DMSO alone or erlotinib dissolved in DMSO (2.5 or 5 μM per well). PBS treated cells served as controls. 72 hours post transduction cells were either visualized under a light microscope or harvested by trypsinization, washed three times with PBS and resuspended in PBS. Harvested cells were subjected to FACS analysis to determine the percentage of GFP positive cells and mean fluorescent intensity.

Western Blotting—H1299 and LNCaP cell lines (5×10⁴ cells/well in 6-well plates) were treated with PBS or transduced with either Ad-luc or Ad-mda7 at an MOI of 2000 viral particles (vp)/cell. Erlotinib, dissolved in DMSO, was administered to both transduced and PBS treated cell cultures at a dose of 5 μM per well. Cells not administered erlotinib (either PBS treated or transduced) served as controls. Cells were harvested 24, 48 or 72 hours after treatment. Total protein was isolated from the harvested cells by adding cell lysis buffer (20 μM HEPES, pH 7.5; 10 mM KCl, 1 mM MgCl₂, 1 mM EDTA, 1 mM DTT, 250 mM sucrose and 1×protease inhibitor). Proteins were separated by SDS polyacrylamide gel electrophoresis and immobilized on nitrocellulose membranes. Membranes were blocked with 5% nonfat dry milk and incubated overnight at 4° C. with the following primary antibodies: beta-actin (Sigma Chemical Co., St. Louis, Mo.); Caspase-3 (Cell Signaling Technology Inc., Beverly, Calif.); MDA-7 (Introgen Therapeutics); Src (Santa Cruz Technology Inc, Beverly, Calif.); and EGFR (Santa Cruz Technology Inc, Beverly, Calif.). Membranes were then washed and incubated with horseradish peroxidase (HRP)-conjugated secondary antibodies for 1 hr at room temperature. Following incubation, the membranes were developed and protein signals detected using enhanced chemiluminescence (ECL) western blotting detection reagents (Amersham Biosciences, Buckinghamshire, UK).

Statistical Analysis—Statistical analysis was performed between control and treated groups, and among the different experimental groups. Comparisons of means were carried out using the Student's t test. Differences with a value of p<0.05 were considered to be statistically significant.

B. Results

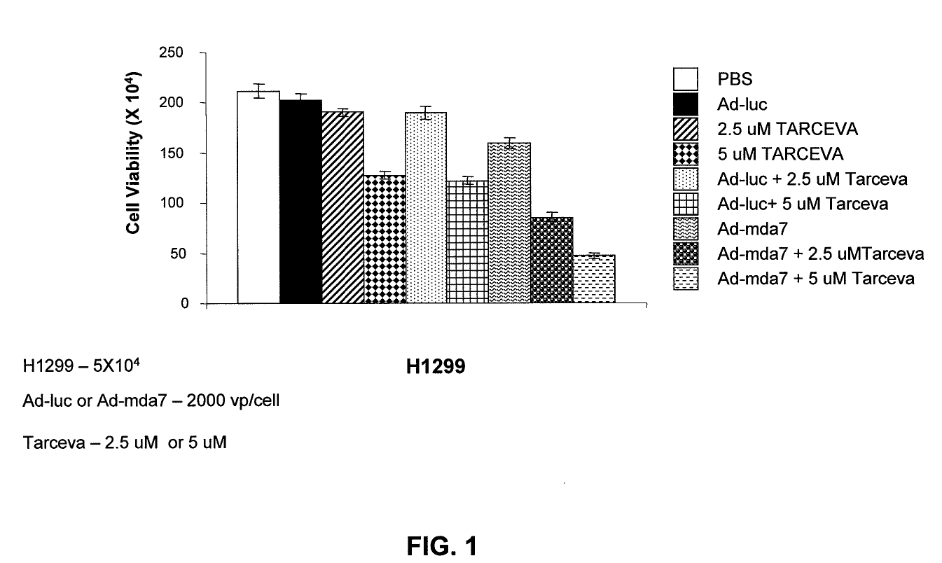

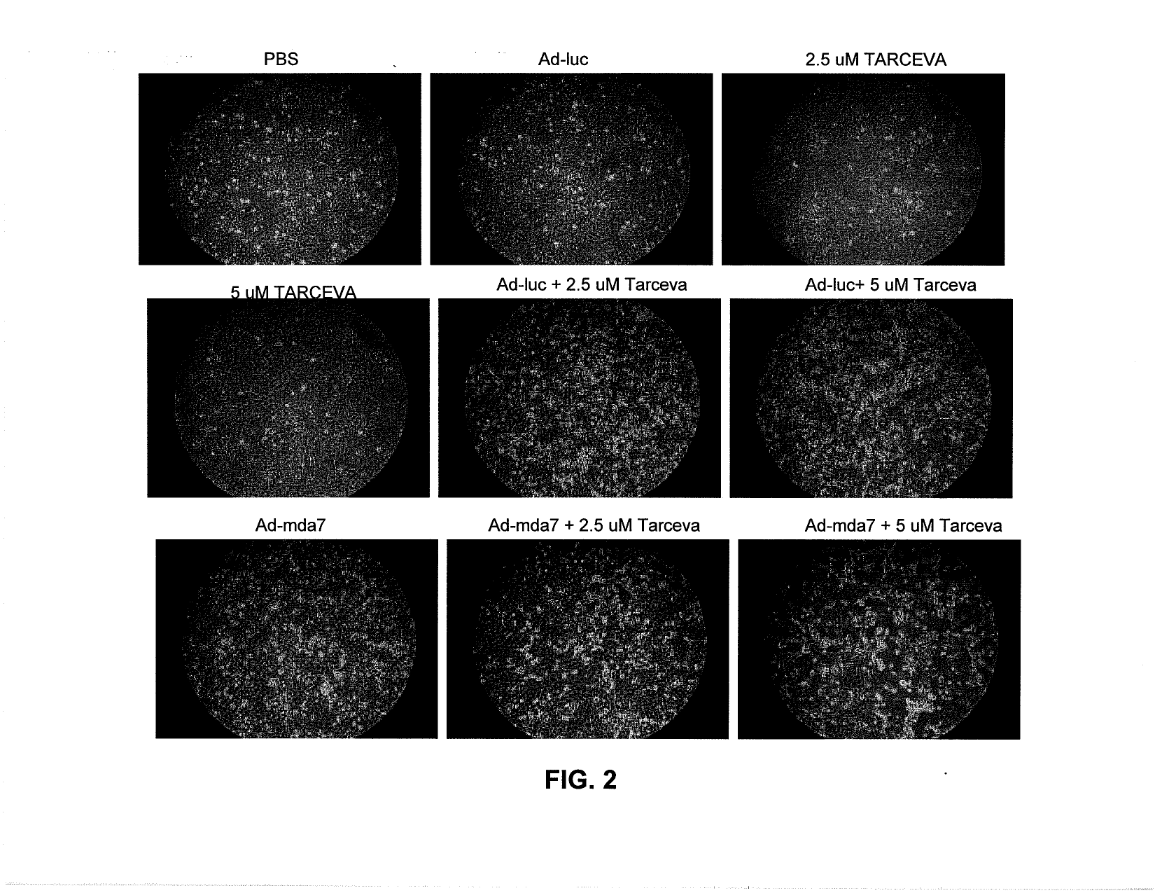

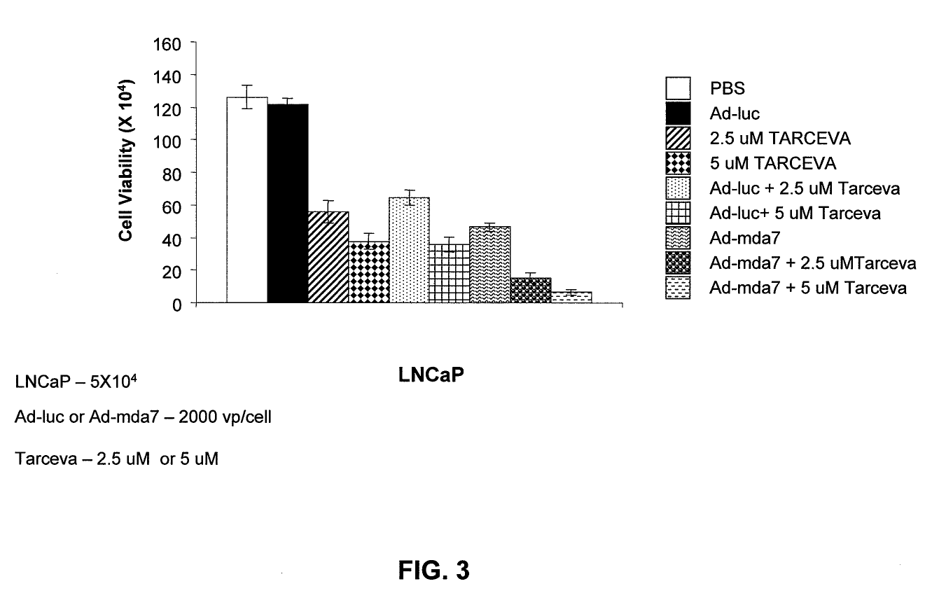

Ad-mda7 and Erlotinib Cotreatment Inhibit Growth of Lung and Prostate Cancer Cells—After being treated with Ad-mda7 and/or erlotinib, cell viability was assessed using trypan blue exclusion assay. The treatment of lung (Hi299) and prostate (LNCaP) tumor cells with Tarceva (2.5 or 5 μM) alone, or with Ad-mda-7 (2000 vp/cell), resulted in the inhibition of cell growth in vitro after 72 h treatment (FIG. 1 and FIG. 3). Furthermore, LNCaP cells appear to demonstrate greater sensitivity to erlotinib at lower dosage levels than do H1299 cells. The treatment of H1299 and prostate LNCaP cells with Ad-mda7 also resulted in the inhibition of cell growth in vivo. When the treatment of H1299 or LNCaP cells involved the combination of erlotinib and Ad-mda7, the result was a significant decrease cell viability (P<0.05) after an incubation of 72 hours as compared to the control cells treated with Ad-luc plus erlotinib. Furthermore, the combination treatment with Ad-mda7 plus erlotinib induced an additive to synergistic effect of growth inhibition in both H1299 and LNCaP cells as compared to Ad-luc plus erlotinib.

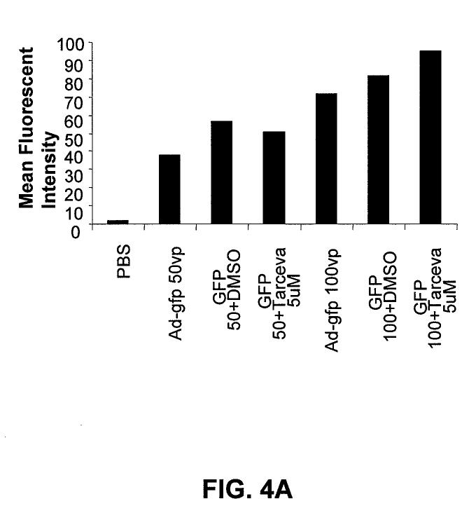

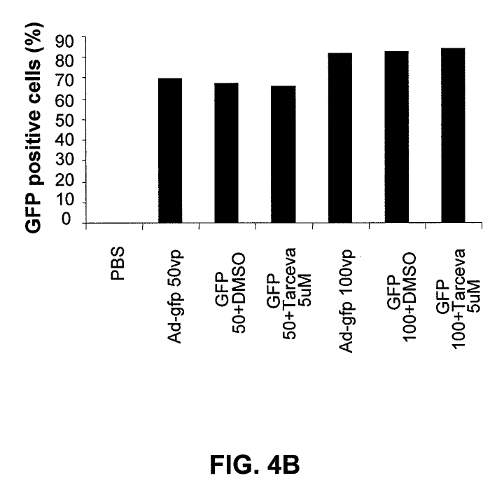

Transduction Efficiency of Adenoviral Vectors Is Independent of Erlotinib Administration—To ensure that the growth inhibition observed in Ad-mda7 plus Tarceva treatment was not simply due to an increase in adenoviral transduction efficiency as a result of erlotinib, transduction efficiency studies were conducted using Ad-GFP. As shown in FIG. 1, tumor (H1299 and LNCaP) cells were treated with Ad-GFP at 50 and 100 vp/cell alone, in the presence of DMSO or with a 5 μM per well dosage of Tarceva. At 72 hours after treatment, cells were harvested and subjected to FACS analysis. While a slight increase in mean fluorescent intensity was in cells treated with Ad-GFP plus erlotinib (FIG. 4A), there was no significant increase in the number of GFP-positive cells treated with this combination (FIG. 4B).

Ad-mda7 and Erlotinib Combination Inhibits Phosphorylation of EGFR—Recent studies have shown that targeting the intracellular kinase activity of EGFR using small molecule inhibitors can result in the inhibition of cancer cell proliferation and the induction of apoptosis (Mellinghoff et al., 2006, Normano et al., 2006 and Sutter et al., 2006). Additionally, Ad-mda7 mediated inhibition of EGFR phosphorylation occurs by inhibiting Src kinase activity and Src-specific phosphorylation sites in the kinase domain of EGFR (Inoue et al., 2005). Furthermore, the Ad-mda7 and Erlotinib combination demonstrated enhanced antitumor activity as evidenced by increased growth inhibitory effects and apoptosis in lung (H1299) and prostate (LNCaP) cancer cells. To elucidate this synergistic effect after the combination of Ad-mda7 and erlotinib, western blots were performed to analyze the levels of EGFR (total and phosphorylated), Src (total and phosphorylated), procaspase-3 and cleaved caspase-3.

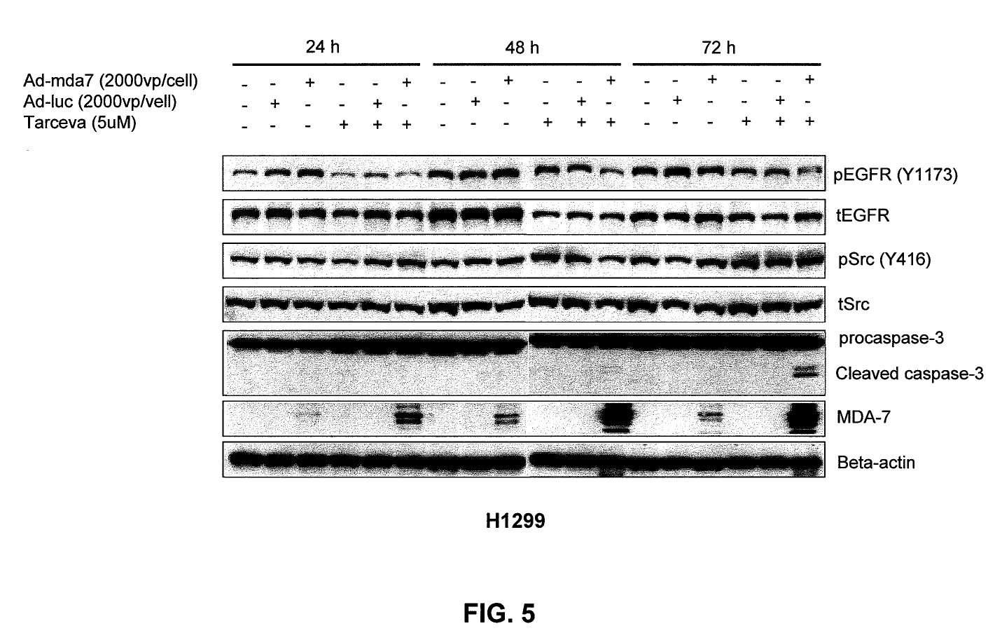

Western blot analysis revealed inhibition of phosphorylation of EGFR in H1299 and LNCaP cancer cells treated with erlotinib, Ad-luc plus erlotinib, and Ad-mda7 plus erlotinib. (FIG. 5) The inhibition of EGFR phosphorylation was greater in those cells treated with the combination of Ad-mda7 and erlotinib. Additionally, the western blot analysis revealed increased caspase-3 activation in Ad-mda7 plus erlotinib treated cells compared to those cells treated with Ad-luc plus erlotinib or Ad-mda7 alone. The caspase-3 activation indicated a correlation with the enhanced growth inhibition of those cells treated with Ad-mda7 plus erlotinib.

The epidermal growth factor receptor (EGFR) is highly expressed in many human cancers and plays an important role in EGF-mediated cell signaling. The binding of epidermal growth factor (EGF) to EGFR results in molecular signaling that favors cell survival. Recent preclinical studies have shown targeting of the extraceullar domain of EGFR using antibodies such as cetuximab or the intracellular kinase domain using small molecule inhibitors such as erlotinib results in tumor growth inhibition. Based on these studies several clinical trials have been conducted testing the antitumor activity of these drugs against several human cancers. However, in majority of these studies treatment with drug alone has shown limited therapeutic effects and clinical response. These studies indicate that additional treatment modalities may need to be incorporated to achieve an effective therapeutic response. The inventors have previously shown that the melanoma differentiation associated gene-7 (MDA-7) protein exerts a potent antitumor activity in vitro and in vivo. Mda-7 gene transfer via Ad-mda7 inhibits tumor growth and induces apoptosis. In the present study the therapeutic effects of erlotinib in combination with Ad-mda7 was assessed using human lung and prostate cancers as a model.

Treatment of lung (H1299) and prostate (LNCaP) tumor cells with Ad-mda7 (1000 -2000 vp/cell) or erlotinib (2.5 or 5 μM) showed inhibition of tumor cell growth in vitro. Treatment of tumor cells with Ad-mda7 plus erlotinib resulted in enhanced growth (P<0.05) inhibition as determined by cell viability assay compared to cells treated with Ad-luc plus erlotinib. Furthermore, combination treatment with Ad-mda7 plus erlotinib induced an additive to synergisitic growth inhibition in both HI 299 and LNCaP tumor cells compared to Ad-luc plus erlotinib treatment. That the growth inhibition observed in Ad-mda7 plus erlotinib treatment was due to increased Ad-mda7 transduction was eliminated by conducting transduction efficiency studies using Ad-GFP. No significant increase in the number of GFP-positive cells was observed when treated with Ad-GFP plus erlotinib compared to cells treated with Ad-GFP alone. Molecular analysis revealed inhibition of phosphorylation of EGFR in tumor cells treated with erlotinib, Ad-luc plus erlotinib and Ad-mda7 plus erlotinib. However, the inhibition of EGFR phosphorylation was greater in Ad-mda7 plus erlotinib-treated cells. These results show that erlotinib and Ad-mda7 may act in concert inhibiting EGFR activity. Ad-mda7-mediated inhibition of EGFR phosphorylation occurs by inhibiting Src kinase activity and the Src-specific phosphorylation site in kinase domain of EGFR. Correlating with enhanced growth inhibition was the increased detection of caspase-3 activation in Ad-mda7 pus erlotinib-treated cells compared to Ad-mda7- and Ad-luc plus erlotinib-treated cells. No significant change in Akt or p44/42 MAPK, targets for erlotinib was observed in any of the treatment groups indicating alternate molecular signaling pathways being inhibited. In conclusion, Ad-mda7 plus erlotinib treatment significantly increases the antitumor activity against human lung and prostate cancer cells.

### Example 2

### The Combination of Lapatinib and AD-MDA7 Synergistically Enhances Cell Death in EGFR Overexpressing Cells

A. Materials and Methods

Cell Lines—MDA-MB-468 cells are human breast cancer cells that overexpress EGFR (HER1). MDA-MB-453 cells are human breast cancer cells that overexpress HER2, and do not overexpress EGFR. Briefly, MDA-MB453 cells and MDA-MB-468 cells were maintained in DMEM supplemented with 10% fetal bovine serum, 10 μM glutamine, 100 units/ml penicillin, 100 mg/ml streptomycin in a 5% CO₂ atmosphere at 37° C.

Recombinant Adenovirus—The recombinant adenoviral vectors used in this example (Ad-mda7 and Ad-luc) are the same as described in Example 1.

Cell Viability Assay—The effect of lapitinib and Ad-mda7 on HER2 overexpressing (MDA-MB-453 and EGFR (HER1) overexpressing (MDA-MB-468) human breast cancer cell lines was evaluated by cell counting after trypan blue (Invitrogen Co., Carlsbad, Calif.) exclusion assay. MDA-MB-453 and MDA-MB-468 cell lines (1×10⁵ cells/well in 6-well plates) were treated with PBS or transduced with either Ad-luc or Ad-mda7 at an MOI of 1000 viral particles (vp)/cell. Lapatinib, dissolved in DMSO, was administered to both transduced and PBS treated cell cultures at a dose of 5 μg/ml per well (2 ml of media per well). Cells not administered lapatinib (either PBS treated or transduced with either adenoviral vector) served as controls. Seventy-two hours after adenoviral vector transduction, cells were trypsinized and an aliquot suspended 1:1 volume with 0.4% trypan blue. Total cell numbers and cell viability counts were assessed using a hemocytometer by light microscopy (Chada et al., 2005).

Apoptosis Assay—MDA-MB-468 cells (1×10⁵ cells per well in a 6 well plate) were treated with either Ad luc, Ad-luc plus lapatinib, Ad-mda7 or Ad-mda7 plus lapatinib at an MOI of 1000 vp per cell. Lapatinib was administered at a concentration of 300 nM per well. Cells were harvested post treatment at day 2, 3 and 4. Hervested cells were fixed, stained with propidium iodide and analyzed via floy cytometry.

B. Results

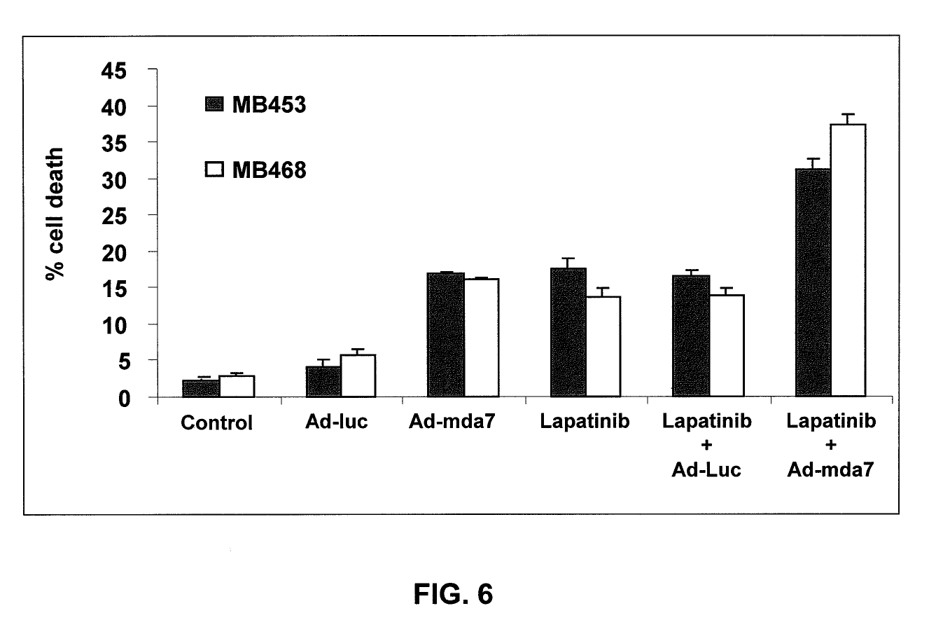

As illustrated in FIG. 6, the treatment of the EGFR overexpressing cancer cell line MDA-MB-468 with the combination of Ad-mda7 and the small molecule EGFR inhibitor lapatinib resulted in a superadditive enhancement of cell killing over either Ad-mda7 or lapatinib alone. While there was some increase in cell death from the combination of lapatinib and Ad-mda7 in MDA-MB-453 cells, which overexpress HER2, these results were not superaddative as that of MDA-MB-468, which overexpresses EGFR (HER1). Additionally, the less than superadditive cell killing effect on MDA-MB-453 cells may be due in some part to the fact that lapatinib can target HER2 as well as EGFR (HER1). Xia et al., 2006. These results are in conformity with the results of erlotinib and Ad-mda7 treatment of EGFR expressing cells.

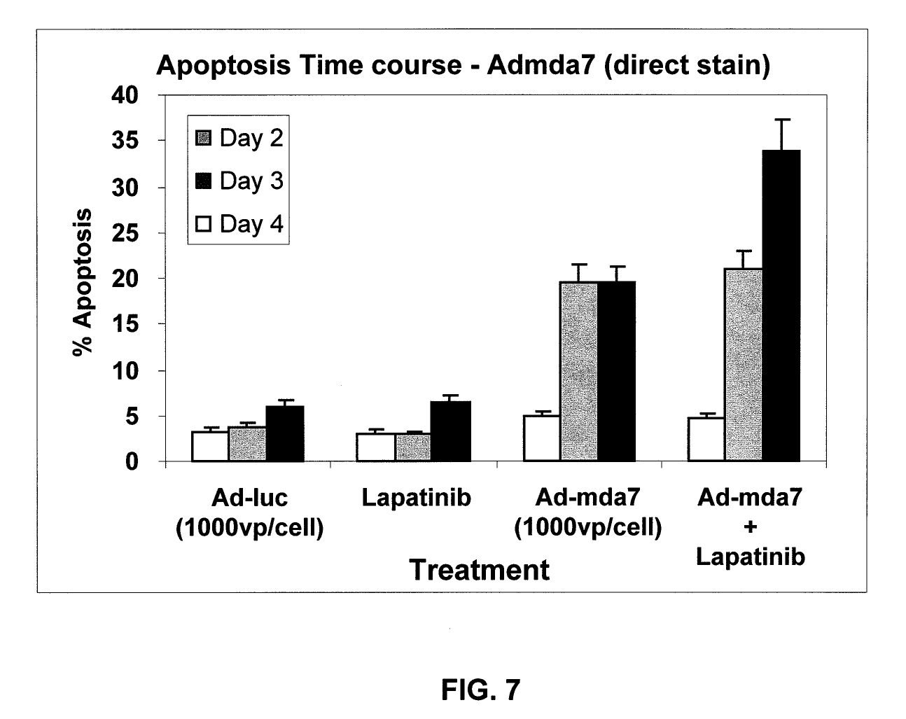

As can be observed from table 3 and FIG. 7, the combination of lapatinib and Ad-MDA7 resulted in an enhancement of apoptosis (35-40% as compared for 5% for Ad-luc or lapatinib treated cells, or 20% for Ad-mda7 alone). Furthermore, the effect of the combination of lapatinib and Ad-mda7 treatment of EGFR overexpressing cancer cells resulted in the highest amount of apoptosis 4 days following initial treatment.

TABLE 3

| | Apoptotic cell percentage | | |
|---|---|---|---|
| Treatment | Day 2 | Day 3 | Day 4 |
| Ad-Luc | 3.32 | 3.77 | 6.02 |
| Lapatinib | 3.07 | 2.98 | 6.52 |
| Ad-mda7 | 5.05 | 19.45 | 19.40 |
| Ad-mda7 + lapatinib | 4.66 | 20.90 | 33.80 |

### Example 3

### The Combination of AD-MDA7 and Cetuximab Treatment of EGFR Overexpressing Cells Results in a Synergistic Increase in Cell Death

A. Materials and Methods

Cell Lines—MCF-7 and MDA-MB-468 cells are human breast cancer cells which do not express and overexpress EGFR, respectively. Briefly, MCF-7 cells and MDA-MB-468 cells were maintained in DMEM supplemented with 10% fetal bovine serum, 10 mM glutamine, 100 units/ml penicillin, 100 mg/ml streptomycin in a 5% CO₂ atmosphere at 37° C.

Recombinant Adenovirus—The recombinant adenoviral vectors used in this example (Ad-mda7 and Ad-luc) are the same as described in Example 1.

Cell Viability Assay—The effect of cetuximab and Ad-mda7 on human non-EGFR expressing (MCF-7) and EGFR overexpressing (MDA-MB-468) human breast cancer cell lines was evaluated by cell counting after trypan blue (Invitrogen Co., Carlsbad, Calif.) exclusion assay. Briefly, MCF-7 and MDA-MB-468 cell lines (1×10⁵ cells/well in 6-well plates) were treated with PBS or transduced with either Ad-luc or Ad-mda7 at an MOI of 1000 viral particles (vp)/cell. Cetuximab, dissolved in PBS, was administered to both transduced and PBS treated cell cultures at a dose of 5 μg/ml per well (2 ml of media per well). Cells not administered cetuximab (either PBS treated or transduced with either adenoviral vector) served as controls. Seventy-two hours after adenoviral vector transduction, cells were trypsinized and an aliquot suspended 1:1 volume with 0.4% trypan blue. Total cell numbers and cell viability counts were assessed using a hemocytometer by light microscopy (Chada et al., 2005).

B. Results

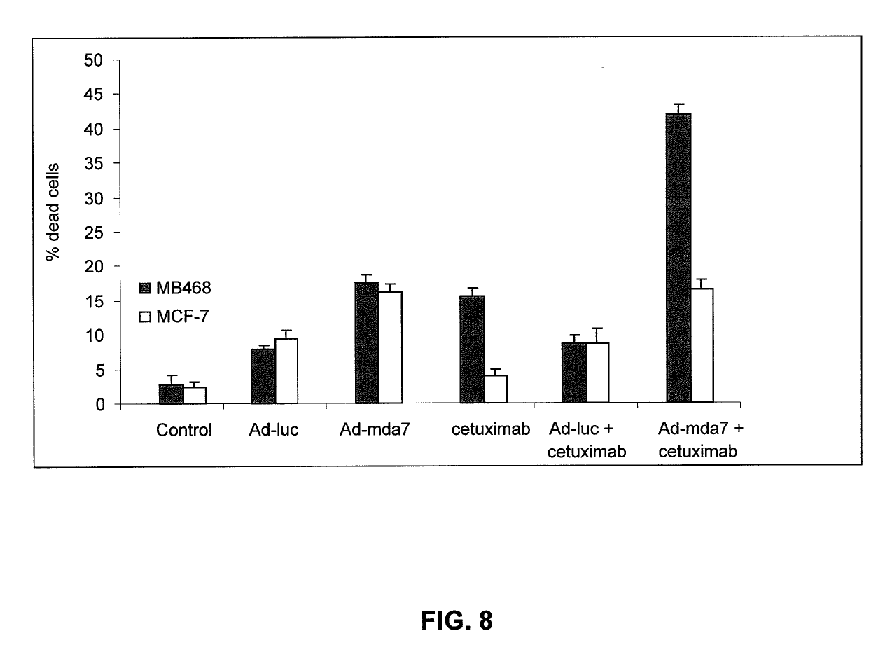

As shown in FIG. 8., the combination of cetuximab and Ad-mda7 resulted in a superadditive enhancement of cell death in EGFR overexpressing cells (MDA-MB-468) over either Ad-mda7 or cetuximab treatment alone. These results correlate well with the synergistic or superadditive cell killing observed using the combination of small molecule EGFR inhibitors such as erlotinib or lapatinib with Ad-mda7 in EGFR overexpressing cells. In contrast to what was observed with the combination of cetuximab and Ad-mda7 in EGFR overexpressing cells, there was little if any increase in cell death using this combination against the non-EGFR expressing MCF-7 cells.

### Example 4

### MDA-7 Inhibits Phosphorylation of Both HER1 and HER2 in Breast Cancer Cells and Potentiates Killing by EGFR Inhibitors

In previous reports, the treatment of HER2+breast cancer cells with the combination Ad-mda7 and trastuzumab (Herceptin®) was shown to result in a synergistic effect resulting in increased cell death. McKenzie et al., 2004. Like trastuzumab, lapatinib exerts an inhibitory effect on HER2, and has been reported to be a dual EGFR (HER1)/HER2 tyrosine kinase inhibitor Xia et al., 2006. See also Example 2, above. Consequently, the inventors herein sought to further explore the effect of the combination of EGFR inhibitors and MDA-7 on HER2+cancer cell lines.

Materials and Methods

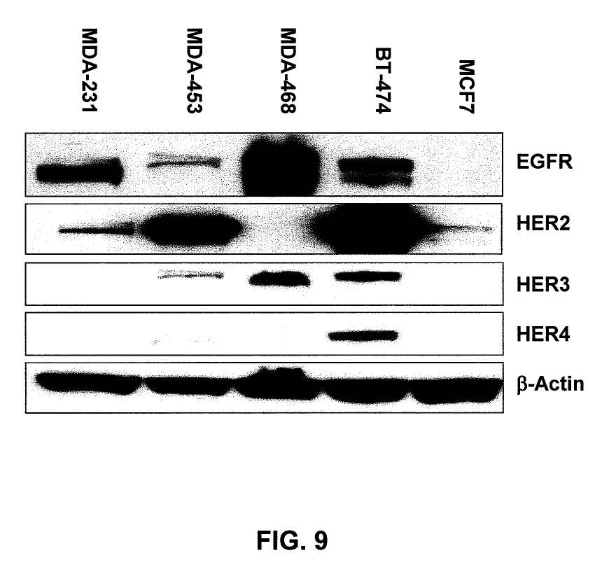

Cell Lines—MDA-MB-231 is a breast cancer cell line that overexpresses EGFR and does express some HER2. MDA-MB-453 overexpresses HER2 and exhibits minimal expression of EGFR. MDA-MB-468 overexpresses EGFR and does not express HER2. BT-474 is a breast cancer cell line that overexpresses both EGFR and HER2. MCF-7 does not overexpress either EGFR or HER2. All cell lines were obtained from American Type Culture Collection (ATCC, Rockville, Md.). Cell lines cells were maintained in DMEM supplemented with 10% fetal bovine serum, 10 mM glutamine, 100 units/ml penicillin, 100 mg/ml streptomycin in a 5% CO₂ atmosphere at 37° C. A western blot demonstrating the relative expression of EGFR and HER2 is shown in FIG. 9, using the western blot techniques described below.

Western Blotting—Total protein was isolated from the harvested cells by adding cell lysis buffer (20 mM HEPES, pH 7.5; 10 mM KCl, 1 mM MgCl₂, 1 mM EDTA, 1 mM DTT, 250 mM sucrose and 1×protease inhibitor). Proteins were separated by SDS polyacrylamide gel electrophoresis and immobilized on nitrocellulose membranes. Membranes were blocked with 5% nonfat dry milk and incubated overnight at 4° C. with the primary antibodies. Primary antibodies optionally included in experiments are anti EGFR, anti HER2, anti HER3, anti HER4, anti p-HER2 (phosphorylated HER2), anti p-EGFR (phosphorylated EGFR), p-ERK and anti beta actin. Membranes were then washed and incubated with horseradish peroxidase (HRP)-conjugated secondary antibodies for 1 hr at room temperature. Following incubation, the membranes were developed and protein signals detected using enhanced chemiluminescence (ECL) western blotting detection reagents (Amersham Biosciences, Buckinghamshire, UK).

Cell Viability Assay—Cells were harvested at optimal times for determining cell death (72 to 96 hours). Cells were trypsinized and an aliquot suspended 1:1 volume with 0.4% trypan blue. Total cell numbers and cell viability counts were assessed using a hemocytometer by light microscopy (Chada et al., 2005).

Recombinant Adenovirus—The recombinant adenoviral vector used in this example (Ad-mda7) is the same as described in Example 1.

Results

Cetuximab Kills EGFR Overexpressing Breast Cancer Cells

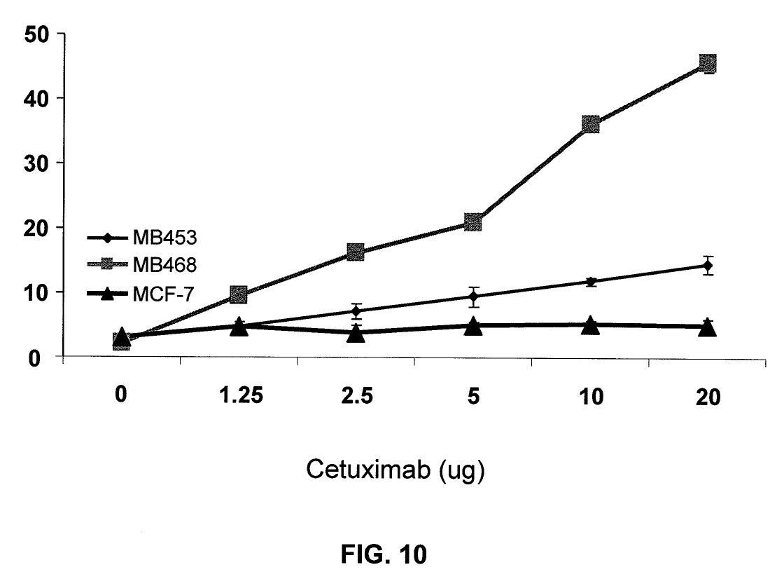

Given the dual specificity of lapatinib, experiments were conducted to determine the efficacy of cetuximab treatment on the cell viability of either EGFR overexpressing or HER2 overexpressing breast cancer cells. Briefly, MDA-MB-453, MDA-MB-468 and MCF-7 cell lines (1×10⁵ cells/well in 6-well plates) were treated with PBS or Cetuximab, dissolved in PBS. Cetuximab, was administered to cell cultures at escalating doses of 1.25, 2.5, 5, 10 and 20 μg/ml per well (2 ml of media per well). Seventy-two hours after treatment, cell viability was determined as described above. As can be seen from FIG. 10, cell viability of both the EGFR overexpressing cell lines (MDA-MB-468) and the HER2 overexpressing cell line (MDA-MB-452) decreased as cetuximab dosage increases. However, the slight increase in cell death in HER2 overexpressing cells may have been the result of some EGFR expression in these cells or some cross reactivity of cetuxumab with HER2. No increase in cell death was seen from the MCF-7 cell line, which does not express either EGFR or HER2.

Ad-mda7 and MDA-7 Inhibit Phosphorylation of EGFR and HER2 in Breast Cancer Cells

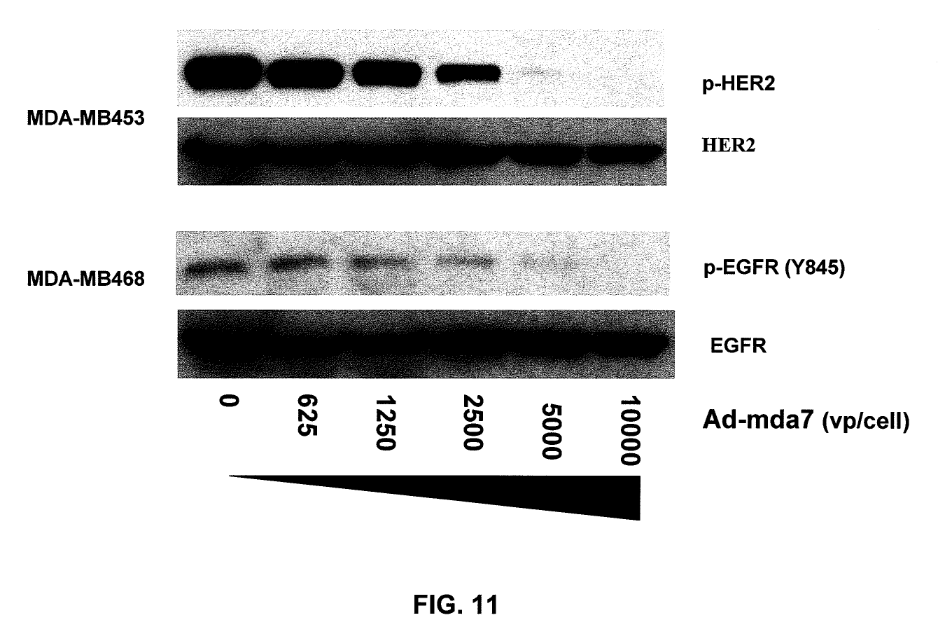

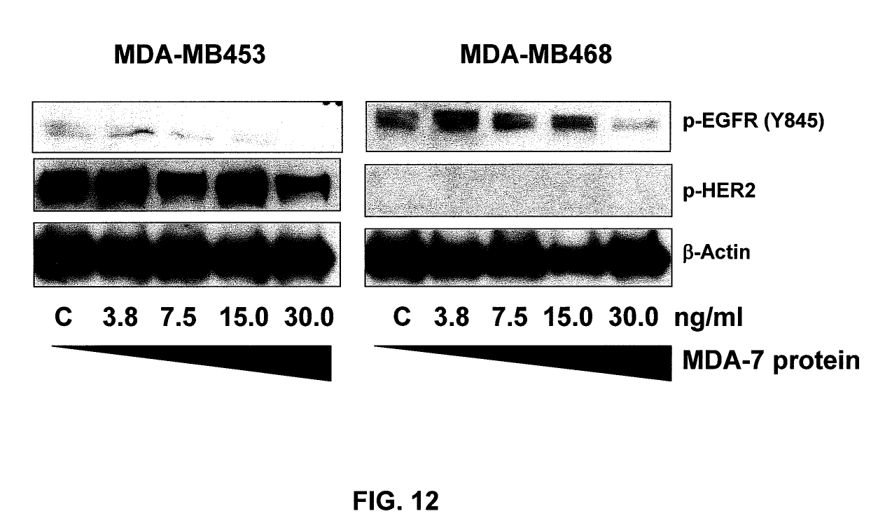

MDA-MB-453 and MDA-MB-468 cell lines (1×10⁵ cells/well in 6-well plates) were treated with PBS or transduced with Ad-mda7 at an escalating MOIs of 625, 1250, 2500, 5000 and 10,000 viral particles (vp)/cell. Cells were harvested 48 or 72 hours after treatment and subjected to sestern blot analysis as described above. As seen in FIG. 11, western blot analysis demonstrates that MDA-7 strongly inhibited the phosphorylation of EGFR as dosage increases. Likewise, MDA-7 inhibited the phosphorylation of HER2 in a dosage dependent manner. In a similar experiment, as shown in FIG. 12, MDA-MB-453 and MDA-MB-468 cell lines were treated with MDA-7 protein in escalating dosage of 3.8, 7.7, 15.0 and 30.0 ng/ml. 48 hours following treatment cells were harvested and subjected to western blot. As indicated in the figure, MDA-7 protein, and not adenoviral vector is the causative agent responsible for the inhibition of phosphorylation of HER2 and EGFR.

Ad-mda7, Lapatinib or Cetuximab Inhibit Phosphorylation of ERK in HER2 Overexpressing Cells

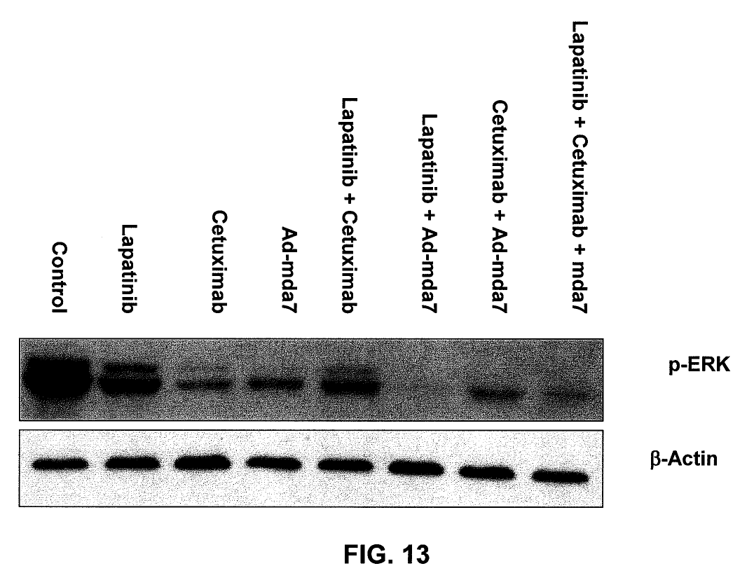

MDA-7 has been reported to regulate the Akt/ERK survival pathways in certain cancers.Bocangel et al., 2006; Mhashilkar et al. Therefore, the inhibition of phosphorylation was deemed to be a logical measure of Ad-mda7 plus EGFR or HER2 inhibitors on cancer cell survival. As shown in FIG. 13, MDA-MB-453 cells (1×10⁵ cells/well in 6-well plates), which overexpress HER2 were treated with PBS or Cetuximab, dissolved in PBS (5 μg/ml per well), or lapatinib dissolved in DMSO (5 μg/ml per well) (2 ml of media per well) or a combination of each. Cells were optionally treated with Ad-mda7 (MOI 1000 vp/cell). As seen in the figure, the combinations of lapatinib+Ad-mda7, cetuxumab+Ad-mda7, and lapatinib+cetuximab+Ad-mda7 resulted in a greater inhibition of phosphorylated ERK than did treatment with any of these reagents alone.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,196,265
U.S. Pat. No. 4,262,017
U.S. Pat. No. 4,415,723
U.S. Pat. No. 4,458,066
U.S. Pat. No. 4,554,101
U.S. Pat. No. 4,797,368
U.S. Pat. No. 4,870,287
U.S. Pat. No. 5,139,941
U.S. Pat. No. 5,179,122
U.S. Pat. No. 5,187,260
U.S. Pat. No. 5,399,363
U.S. Pat. No. 5,466,468
U.S. Pat. No. 5,543,158
U.S. Pat. No. 5,641,515
U.S. Pat. No. 5,739,169
U.S. Pat. No. 5,760,395
U.S. Pat. No. 5,795,715
U.S. Pat. No. 5,801,005
U.S. Pat. No. 5,824,311
U.S. Pat. No. 5,830,880
U.S. Pat. No. 5,846,225
U.S. Pat. No. 5,846,233
U.S. Pat. No. 5,846,945
U.S. Pat. No. 6,858,227
U.S. Pat. No. 5,889,136
U.S. application Ser. No. 09/575,473
U.S. application Ser. No. 09/615,154
U.S. application Ser. No. 10/017,472
U.S. application Ser. No. 10/378,590
U.S. application Ser. No. 10/391,068
U.S. application Ser. No. 10/791,692
U.S. application Ser. No. 10/791,692
U.S. application Ser. No. 60/661,680
U.S. Pub. No. 20030147966
U.S. Pub. No. 20030223938
U.S. Pub. No. 20050143336
Agnew et al., J. Chromatogr. B Biomed. Sci. Appl., 755(1-2):237-243, 2001.
Aksentijevich et al., Hum. Gene Ther., 7(9):1111-1122, 1996.
Alberts et al., J. Cell. Biochem. Supp., (22):18-23, 1995.
Amat et al., Brit. J. Cancer, 88:1339-1345, 2003.
Angel et al., Cell, 49:729, 1987b.
Angel et al., Mol. Cell. Biol., 7:2256, 1987a.
Arap et al., Cancer Res., 55(6):1351-1354, 1995.
Atchison and Perry, Cell, 46:253, 1986.
Atchison and Perry, Cell, 48:121, 1987.
Austin-Ward and Villaseca, Revista Medica de Chile, 126(7):838-845, 1998
Ausubel et al., In: Current Protocols in Molecular Biology, John, Wiley & Sons, Inc, New York, 1996.
Bajorin et al., J. Clin. Oncol., 6(5):786-792, 1988.
Bakhshi et al., Cell, 41(3):899-906, 1985
Bakin et al., J. Biol. Chem., 275:36803-36810, 2000.
Banerji et al., Cell, 27(2 Pt 1):299-308, 1981.
Banerji et al, Cell, 33(3):729-740, 1983.
Barber, Death Differ., 8:113-126, 2001.
Basu et al, Mol. Cancer Res., 2:632-642, 2004.
Bateman and Uccellini, J. Pharm. Pharmacol., 36(7):461-464, 1984
Bedi et al., Cancer Res., 55(9):1811-1816, 1995.
Benoit et al., Biochem. Cell Biol., 82(6):719-727, 2004.
Benoit et al., Oncogene, 23:1631-1635, 2004.
Berkhout et al., Cell, 59:273-282, 1989.
Birringer et al., Br. J. Cancer, 88(12):1948-55, 2003.
Blanar et al., EMBO J., 8:1139, 1989.
Bocangel et al., Cancer Gene Ther., 13(10):958-68, 2006.
Bodine and Ley, EMBO J., 6:2997, 1987.
Bonnadona and Karnofsky, J. Clin. Oncol., 7:1380-1397, 1989.
Bonvini et al., Cancer Res., 61:1671-1677, 2001.
Boshart et al., Cell, 41:521, 1985.
Bosher and Labouesse, Nat. Cell Biol., 2:E31-E36, 2000.
Bosze et al., EMBO J., 5(7):1615-1623, 1986
Boyd et al., Clin. Cancer Res., 10:1545-1555, 2004.
Braddock et al., Cell, 58:269, 1989.
Bromann et al., Oncogene, 23:7957-7968, 2004.
Bukowski et al., Clin. Cancer Res., 4(10):2337-2347, 1998.
Bulla and Siddiqui, J. Virol., 62:1437, 1986.
Burow et al., Biochem. Biophys. Res. Commun., 271:342-345, 2000.
Caldas et al., Nat. Genet., 8(1):27-32, 1994.
Campbell and Villarreal, Mol. Cell. Biol., 8:1993, 1988.
Campbell et al., Cancer Res., 64(21):7678-7681, 2004.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Campere and Tilghman, Genes and Dev., 3:537, 1989.
Campo et al., Nature, 303:77, 1983.
Caplen, et al., Gene, 252:95-105, 2000.
Caudell et al., J. Immunol., 168(12):6041-6046, 2002.
Celander et al., J. Virology, 62:1314, 1988.
Chada et al., Int. Immunopharmacol., 4(5):649-667, 2004.
Chada et al., Int. Immunopharmacol., 4:649-667, 2004.
Chada et al., Mol. Ther., 10(6):1085-1095, 2004.
Chada et al., Mol. Ther., 11(5):724-733, 2005
Chada et al., Mol. Ther., 7:S446, 2003.
Chandler et al., Cell, 33:489, 1983.
Chang et al., Mol. Cell. Biol., 9:2153, 1989.
Chatterjee et al., Proc. Natl. Acad. Sci. USA, 86:9114, 1989.
Cheng et al., Cancer Res., 54(21):5547-5551, 1994.
Choi et al., Cell, 53:519, 1988.
Christodoulides et al., Microbiology, 144(Pt 11):3027-3037, 1998.
Clark et al., Hum. Gene Ther., 6(10):1329-1341, 1995.
Cleary and Sklar, Proc. Natl. Acad. Sci. USA, (21):7439-7443, 1985.
Cleary et al., J. Exp. Med., 164(1):315-320, 1986.
Coffin, In: Virology, Fields et al (Eds.), Raven Press, NY, 1437-1500, 1990.
Cohen et al., J. Cell. Physiol., 5:75, 1987.
Costa et al., Mol. Cell. Biol, 8:81, 1988.
Craven et al., Surg. Oncol., 12(1):39-49, 2003.
Cripe et al., EMBO J., 6:3745, 1987.
Culotta and Hamer, Mol. Cell. Biol., 9:1376, 1989.
Dandolo et al., J. Virology, 47:55-64, 1983.
Davidson et al., J. Immunother., 21(5):389-398, 1998.
De Villiers et al., Nature, 312(5991):242-246, 1984.
Denkert et al., Clin. Breast Cancer, 4(6):428-433, 2004.
Deschamps et al., Science, 230:1174-1177, 1985.
Dietary Reference Intakes for Vitamin C, Vitamin E, Selenium, and Carotenoids from the Panel on Dietary Antioxidants and Related Compounds, Institutue of Medicine, Food and Nutrition Board, National Academy Press Washington, D.C., 2000.
Dillman, Cancer Biother. Radiopharm., 14(1):5-10, 1999.
Donze et al., EMBO J., 20:3771-3780, 2001.
DuBois et al., Gastroenterology, (25):773-791, 1996.
Earnest et al., J. Cell Biochem. Suppl., 16I:156-166, 1992.
Edbrooke et al., Mol. Cell. Biol., 9:1908, 1989.
Edlund et al., Science, 230:912-916, 1985.
Ekmekcioglu et al., Int. J. Cancer, 94(1):54-59, 2001.
El-Rayes et al., Mol. Cancer Ther., 3:1421-1426, 2004.
Elbashir et al., Genes Dev., 15:188-200, 2001.
Enmon et al., Cancer Res., 63:8393-8399, 2003.
Fariss et al., Cancer Res., 54:3346-3351, 1994.
Felgner et al., Proc. Natl. Acad. Sci. USA, 84(21):7413-7417, 1987.
Feng and Holland, Nature, 334:6178, 1988.
Firak and Subramanian, Mol. Cell. Biol., 6:3667, 1986.
Fire et al., Nature, 391:806-811, 1998.
Fisher et al., J. Natl. Cancer Inst., 89(22):1673-1682, 1997.
Flotte et al., Am. J. Respir. Cell Mol. Biol., 7(3):349-356, 1992.
Flotte et al., Proc. Natl. Acad. Sci. USA, 90(22):10613-10617, 1993.
Flotte, et al., Gene Ther., 2(1):29-37, 1995.
Foecking and Hofstetter, Gene, 45(1):101-105, 1986.
Fraley et al., Proc. Natl. Acad. Sci. USA, 76:3348-3352, 1979.
Freifelder, In: Physical Biochemistry Applications to Biochemistry and Molecular Biology, 2nd Ed. Wm. Freeman and Co., NY, 1982.
Freshney, In: Animal Cell Culture, A Practical Approach, 2^{nd} Ed., Oxford Press, UK, 1992.
Fry, Breast Cancer Res., 3:304-312, 2001.
Fujita et al., Cell, 49:357, 1987.
Gabizon et al., Cancer Res., 50(19):6371-6378, 1990.
Gaensler et al., Nat. Biotechnol., 17:1188-1192, 1999.
Gann et al., J. Natl. Cancer Inst., 85:1220-1224, 1993.
Garner et al., Cochrane Database Syst Rev., (4):CD003831, 2002.
Gerber et al., J. Biol. Chem., 273:30336-30343, 1998. Celander and Haseltine, J. Virology, 61:269, 1987.
Gewies et al., Cancer Res., 60:2163-2168, 2000.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Giovannucci et al., Ann. Intern. Med., 121:241-246, 1994.
Gloss et al., EMBO J., 6:3735, 1987.
Godbout et al., Mol. Cell. Biol., 8:1169, 1988.
Goding, In: Monoclonal Antibodies: Principles and Practice, 2d ed., Academic Press, Orlando, Fla., pp 60-61, 71-74, 1986.
Goodboum and Maniatis, Proc. Natl. Acad. Sci. USA, 85:1447, 1988.
Goodboum et al., Cell, 45:601, 1986.
Greenberg et al., J. Natl. Cancer Inst., 85:912-916, 1993.
Greene et al., Immunology Today, 10:272, 1989
Grishok et al., Science, 287:2494-2497, 2000.
Grosschedl and Baltimore, Cell, 41:885, 1985.
Hanibuchi et al., Int. J. Cancer, 78(4):480-485, 1998.
Hanif et al., Biochemical Pharmacology, (52):237-245, 1996.
Harlow and Lane, In: Antibodies: A Laboratory Manual, Cold Spring Harbor Laboratory Press, NY, 1988.
Haslinger and Karin, Proc. Natl. Acad. Sci. USA, 82:8572, 1985.
Hauber and Cullen, J. Virology, 62:673, 1988.
Hellstrand et al., Acta Oncol., 37(4):347-353, 1998.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Hinz and Brune, Wien Klin Wochenschr., 111 (3): 103-12, 1999.
Hirochika et al.,J. Virol., 61:2599, 1987.
Hirsch et al., Mol Cell. Biol., 10:1959, 1990.
Holbrook et al., Virology, 157:211, 1987.
Horlick and Benfield, Mol. Cell. Biol., 9:2396, 1989.
Howe et al., Cancer Res., 62:5405-5407, 2002.
Howe et al., Endocr. Relat. Cancer, 8:97-114, 2001.
Hsu et al., J. Biol. Chem., 275:11397-11403, 2000.
Hu et al.,J. Antibiot. (Tokyo), 57(7):421-428, 2004.
Huang et al., Cell, 27:245, 1981.
Hug et al., Mol. Cell. Biol., 8:3065, 1988.
Hui and Hashimoto, Infect. Immun., 66(11):5329-5336, 1998.
Hussussian et al., Nat. Genet., 8(1):15-21, 1994.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol Cell. Biol., 4:875, 1984.
Inoue et al., Mol. Ther., 12:707-715,2005.
Inouye and Inouye, Nucleic Acids Res., 13:3101-3109, 1985.
Irby and Yeatman, Oncogene, 19:5636-5642, 2000.
Irie and Morton, Proc. Natl. Acad. Sci. USA, 83(22):8694-8698, 1986.
Irie et al., Lancet., 1(8641):786-787, 1989.
Jagus et al., Int. J. Biochem. Cell. Biol., 31:123-138, 1999.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jemal et al., Cancer J. Clin., 52:23-47, 2002.
Jiang et al., Proc. Natl. Acad. Sci. USA, 93(17):9160-9165, 1996.
Jiang et al., Proc. Natl. Acad. Sci. USA, 93:9160-9165, 1996.
Johnson et al., Mol. Cell. Biol., 9:3393, 1989.
Ju et al., Gene Ther., 7(19):1672-1679, 2000.
Kabat et al., In: Sequences of Proteins of Immunological Interest, U.S. Department of Health and Human Services, Washington, D.C., 1987.
Kadesch and Berg, Mol. Cell. Biol., 6:2593, 1986.
Kamal et al., Nature.,425:407-410, 2003.
Kamb et al., Nat. Genet., 8(1):23-26, 1994.
Kaneda et al., Science, 243:375-378, 1989.
Kaplitt et al., Nat Genet., 8(2):148-154, 1994.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Kato et al., J. Biol. Chem., 266:3361-3364, 1991.
Kawamori et al., Cancer Res 58(3): 409-12, 1998.
Kawamoto et al., Mol. Cell. Biol., 8:267, 1988.
Kerr et al, Br. J. Cancer, 26(4):239-257, 1972.
Ketting et al., Cell, 99:133-141, 1999.
Kiledjian et al., Mol. Cell. Biol., 8:145, 1988.
Kismet et al., Cancer Detect. Prev., 10(6):589-601, 2004.
Klamut et al., Mol. Cell. Biol, 10:193, 1990.
Kline et al., In: Proceeding of the International Conference on Nutrition and Cancer, Prasad and Cole (Eds.), Amsterdam: IOS Press, 37-53, 1998.
Kline et al., J. Mammary Gland Biol. Neoplasia 8:91-102, 2003.
Kline et al., J. Nutr., 131: 161 S-163S, 2001.
Knueferrnann et al., Oncogene, 22:3205-3212, 2003.
Koch et al., Mol. Cell. Biol., 9:303, 1989.
Koch, et al., Rev. Physiol. Biochem. Pharmacol. 146:55-94, 2003.
Koehne and Dubois, Semin. Oncol., 31:12-21, 2004.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kulp et al., Cancer Res., 64:1444-1451, 2004.
Kunz et al., Nucl. Acids Res., 17:1121, 1989.
Kyte and Doolittle, J. Mol. Biol., 157(1):105-132, 1982.
Ladenheim et al., Gastroenterology, 108:1083-1087, 1995.
LaFace et al., Virology, 162(2):483-486, 1988.
Lanza et al., Arch. Intern. Med., 155:1371-1377, 1995.
Larsen et al., Proc Natl. Acad. Sci. USA., 83:8283, 1986.
Laspia et al., Cell, 59:283, 1989.
Latimer et al., Mol. Cell Biol., 10:760, 1990.
Laughlin et al., J. Virol., 60(2):515-524, 1986.
Lawson et al., Mol. Cancer Ther., 2:437-444, 2003.
Le Brazadec et al., J. Med. Chem., 47(15):3865-3873, 2004.
Le et al., J. Biol. Chem., 280(3):2092-2104, 2005.
Lebkowski et al., Mol. Cell. Biol., 8(10):3988-3996, 1988.
Lee et al., Nature, 294:228, 1981.
Lee et al., Nucleic Acids Res., 12:4191-206, 1984.
Leng et al., Hepatology, 38:756-768, 2003.
Levinson et al., Nature, 295:79, 1982.
Lin et al., Mol. Cell. Biol., 10:850, 1990.

Lin and Avery, Nature, 402:128-129, 1999.
Liu et al., Am. J. Clin. Oncol., 26:S103-109, 2003.
Liu et al., Cancer Res., 55(14):3117-3122, 1995.
Lupulescu, Cancer Detect. Prev., 20(6):634-637, 1996.
Luria et al., EMBO J., 6:3307, 1987.
Lusky and Botchan, Proc. Natl. Acad. Sci. USA, 83:3609, 1986.
Lusky et al., Mol. Cell. Biol, 3:1108, 1983.
Macejak and Samow, Nature, 353:90-94, 1991.
Majors and Varmus, Proc. Natl. Acad. Sci. USA, 80:5866, 1983.
Malafa and Neitzel, J. Surg. Res., 93:163-170, 2000.
Malafa et al., Surgery, 131:85-91, 2002.
Maloney and Workman, Expert Opin. Biol. Ther., 2:3-24, 2002.
Mandler et al., Bioconjug. Chem., 13(4):786-791, 2002.
Mandler et al., Cancer Res., 64(4):1460-1467, 2004.
Mann et al., Cell, 33:153-159, 1983.
Marsters et al., Recent Prog. Horm. Res., 54:225-234, 1999.
Marte and Downward, Trends Biochem Sci., 22:355-358, 1997.
McCarty et al., J. Virol., 65(6):2936-2945, 1991.
McKenzie et al., Surgery, 136:437-442, 2004.
McLaughlin et al., J. Virol., 62(6):1963-1973, 1988.
McNeall et al., Gene, 76:81, 1989.
Mellinghoff et al., N. Engl. J. Med., 353:2012-2024, 2006.
Mhashilkar et al., Mol. Med., 7(4):271-282, 2001.
Mhashilkar et al., Mol. Ther., 8:207-219, 2003.
Miksicek et al., Cell, 46:203, 1986.
Mitchell et al., Ann. NY Acad. Sci., 690:153-166, 1993.
Mitchell et al., J. Clin. Oncol., 8(5):856-869, 1990.
Montgomery et al., Proc. Natl. Acad. Sci. USA., 95:15502-15507, 1998.
Mordacq and Linzer, Genes and Dev., 3:760, 1989.
Moreau et al., Nucl. Acids Res., 9:6047, 1981.
Mori et al., Cancer Res., 54(13):3396-3397, 1994.
Morton et al., Arch. Surg., 127:392-399, 1992.
Muesing et al., Cell, 48:691, 1987.
Muscat et al., Cancer, 74:1847-1854, 1994.
Muzyczka, Curr. Topics Microbiol. Immunol., 158:97-129, 1992.
Narisawa et al., Cancer Res., 41(5):1954-1957, 1981.
Neckers, Trends Mol. Med., 8(4 Suppl):S55-S56, 2002.
Neuzil et al., Br. J. Cancer, 84:87-89, 2000.
Neuzil et al., FASEB J., 15:403-415, 2001.
Ng et al., Nuc. Acids Res., 17:601, 1989.
Nicholson and Anderson, Cell Signal, 14(5):381-395, 2002.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nishikawa et al., Mol. Ther., 9:818-828, 2004.
Nishikawa et al., Oncogene, 23:7125-7131, 2004.
Nobri et al., Nature (London), 368:753-756, 1995.
Normano et al., J. Cell Physiol., 207:420-427, 2006.
Ohi et al., Gene, 89(2):279-282, 1990.
Okamoto et al., Proc. Natl. Acad. Sci. USA, 91(23):11045-11049, 1994.
Ondek et al., EMBO J., 6:1017, 1987.
Orlow et al., Cancer Res, 54(11):2848-2851, 1994.
Ornitz et al., Mol. Cell. Biol., 7:3466, 1987.
Palmiter et al., Nature, 300:611, 1982.
Paskind et al., Virology, 67:242-248, 1975.
Pataer et al., Cancer Res., 62:2239-2243, 2002.
Patel et al., Chem. Biol, 11(12):1625-1633, 2004.

PCT Appln. WO 01/36646

PCT Appln. WO 01/68836
PCT Appln. WO 00/05356
PCT Appln. WO 00/26368

PCT Appln. WO 00/44914

PCT Appln. WO 99/32619
PCT Appln. WO 98/07408
PCT Appln. WO 98/28425
Pech et al., Mol. Cell. Biol., 9:396, 1989.
Pegram et al., J. Natl. Cancer Inst., 96:739-749, 2004.
Pelletier and Sonenberg, Nature, 334(6180):320-325, 1988.
Perez-Stable and Constantini, Mol. Cell. Biol., 10:1116, 1990.
Pestka et al., Annu. Rev. Immunol., 22:929-979, 2004.
Peto et al., Lancet, 355:1822, 2000.
Piazza et al., Cancer Res., (55):311 3116, 1995.
Piazza et al., Cancer Res., (57):2452-2459, 1997b.
Piazza et al., Cancer Res., (57):2909-2915, 1997a.
Picard and Schaffner, Nature, 307:83, 1984.
Pietras et al., Oncogene, 17(17):2235-2249, 1998.
Pinkert et al., Genes and Dev., 1:268, 1987.
Pisani et al., Int. J. Cancer, 83:18-29; 870-873, 1999.
Pollard and Luckert, Cancer Res., 49:6471-6473, 1989.
Ponta et al., Proc. Natl. Acad. Sci. USA, 82:1020, 1985.
Porton et al., Mol. Cell. Biol., 10:1076, 1990.
Prasad and Edwards-Prasad, J. Am. Coll. Nutr., 11:487-500, 1992.
Prasad and Edwards-Prasad., Cancer Res., 42:550-554, 1982.
Qin et al., Proc. Natl. Acad. Sci. USA, 95(24):14411-14416, 1998.
Queen and Baltimore, Cell, 35:741, 1983.
Quinn et al., Mol. Cell. Biol., 9:4713, 1989.
Rahmani et al., Cancer Res., 63:8420-8427, 2003.
Ramesh et al., Mol. Ther., 3:337-350, 2001.
Ramesh et al., Cancer Res., 63:5105-5113, 2003.
Ramesh et al., DNA Cell Biol., 23:850-857, 2004.
Rao et al., Cancer Res., 55(7):1464-1472, 1995.
Ravindranath and Morton, Intern. Rev. Immunol., 7: 303-329, 1991.
Reddy et al., Cancer Res., (50):2562-2568, 1990.
Reddy et al., Cancer Res., 47:5340-5346, 1987.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Resendez Jr. et al., Mol. Cell. Biol., 8:4579, 1988.
Ripe et al., Mol. Cell. Biol., 9:2224, 1989.
Ritland and Gendler, Carcinogenesis, 20(1): 51-58, 1999.
Rittling et al., Nuc. Acids Res., 17:1619, 1989.
Rosen et al., Cell, 41:813, 1988.
Rosenberg et al., Ann. Surg. 210(4):474-548, 1989.
Rosenberg et al., Nature Med., 4:321-327, 1998.
Ross et al., Oncologist, 8(4):307-25, 2003.
Saeki et al., Gene Ther., 7:2051-2057, 2000.
Saeki et al., Oncogene, 21:4558-4566, 2002.
Sakai et al., Genes and Dev., 2:1144, 1988.
Sambrook et al., In: Molecular cloning, Cold Spring Harbor Laboratory Press, Cold Spring Harbor, NY, 2001.
Samulski et al., J. Virol., 63:3822-3828, 1989.
Sarkar et al., Biotechniques Suppl., 30-39, 2002.
Satake et al., J. Virology, 62:970, 1988.
Sausville et al., Curr. Cancer Drug Targets, 3:377-383, 2003.
Schaefer et al., Cell Immunol., 214:110-122, 2001.
Schaffner et al., J. Mol. Biol, 201:81, 1988.
Schulte and Neckers, Cancer Chemother. Pharmacol., 42(4):273-279, 1998.
Schwartz and Shklar, J. Oral Maxillofac. Surg., 50:367-373, 1992.
Searle et al., Mol. Cell. Biol., 5:1480, 1985.
Serrano et al., Nature, 366:704-707, 1993.
Serrano et al., Science, 267(5195):249-252, 1995.
Sharp, Genes Dev., 13:139-141, 1999.
Sharp and Marciniak, Cell, 59:229, 1989.
Sharp and Zamore, Science, 287:2431-2433, 2000.
Shaul and Ben-Levy, EMBO J., 6:1913, 1987.
Shelling and Smith, Gene Therapy, 1:165-169, 1994.
Sherman et al., Mol. Cell. Biol., 9:50, 1989.
Simstein et al., Exp. Biol. Med., 228(9):995-1003, 2003.
Singh and Lippman, Oncology (Huntingt), 12(12):1787-800, 1998.
Singh and Reddy, Annals. NY Acad. Sci., (768):205-209, 1995.
Singh et al., Carcinogenesis, (15):1317-1323, 1994.
Sleigh and Lockett, J. EMBO, 4:3831, 1985.
Smith et al., Cancer Chemother. Pharmacol., 54(6):475-486, 2004.
Smyth-Templeton et al., DNA Cell Biol., 21(12):857-867, 1997.
Solit et al., Cancer Res., 63:213921-213944, 2003.
Solodin et al., Biochemistry, 34(41):13537-13544, 1995.
Soo et al., J. Cell. Biochem., 74(1):1-10, 1999.
Spalholzet al., Cell, 42:183, 1985.
Spandau and Lee, J. Virology, 62:427, 1988.
Spandidos and Wilkie, EMBO J., 2:1193, 1983.
Steinbach et al., N. Engl. J. Med., 342:1946-1952, 2000.
Stephens and Hentschel, Biochem. J., 248:1, 1987.
Stuart et al., Nature, 317:828, 1985.
Su et al., Cancer Res., 58, 2339-2342, 1998.
Su et al., Proc. Natl. Acad. Sci. USA, 95:14400-14405, 1998.
Sullivan and Peterlin, Mol. Cell. Biol., 7:3315, 1987.
Sutter et al., Int. J. Cancer, 118: 1814-1822, 2006.
Swartzendruber and Lehman, J. Cell. Physiology, 85:179, 1975.

Tabara et al., Cell, 99:123-132, 1999.
Takebe et al., Mol. Cell. Biol., 8:466, 1988.
Tantivejkul et al., Breast Cancer Res. Treat., 79:301-312, 2003.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Taylor et al., Science, 285:107-110, 1999.
Temin, In: Gene Transfer, Kucherlapati (Ed.), NY, Plenum Press, 149-188, 1986.
Templeton et al., Nat. Biotechnol., 15:647-652, 1997.
Thierry et al., Proc. Natl Acad. Sci. USA, 92(21):9742-9746, 1995.
Thiesen et al., J. Virology, 62:614, 1988.
Thompson et al., J. Natl. Cancer Inst., (87):125-1260, 1995.
Thun et al., N. Engl. J. Med., 325:1593-1596, 1991.
Tian et al., Bioorg. Med. Chem., 12(20):5317-5329, 2004.
Traber et al., Am. J. Clin. Nutr., 48,605-611, 1988.
Traber, Biofactors, 10(2-3):115-120, 1999.
Tratschin et al., Mol. Cell. Biol., 4:2072-2081, 1984.
Tratschin et al., Mol. Cell. Biol., 5:3258-3260, 1985.
Treisman, Cell, 42:889, 1985.
Tronche et al., Mol. Biol. Med., 7:173, 1990.
Trudel and Constantini, Genes and Dev., 6:954, 1987.
Tsujimoto and Croce, Proc. Natl. Acad. Sci. USA, 83(14):5214-5218, 1986.
Tsujimoto et al., Science, 228(4706):1440-1443, 1985.
Tsukamoto et al., Nat. Genet., 9(3):243-248, 1995.
Tyndell et al., Nuc. Acids. Res., 9:6231, 1981.
Vane, and Botting, Sem. in Arthritis and Rheumatism, (26):2-10, 1997.
Vannice and Levinson, J. Virology, 62:1305, 1988.
Vasseur et al., Proc Natl. Acad. Sci. USA, 77:1068, 1980.
Vorburger et al., Oncogene, 21:6278-6288, 2002.
Walsh et al., J. Clin. Invest, 94:1440-1448, 1994.
Walsh et al., J. Clin. Invest., 94(4):1440-1448, 1994.
Walter and Johnson, Annu. Rev. Cell Biol. 10:87-119, 1994.
Wang and Calame, Cell, 47:241, 1986.
Wang et al., J. Biol. Chem., 277(9):7341-7347, 2002.
Weber et al., Cell, 36:983, 1984.
Weber et al., Clin. Cancer Res., 8:863-869, 2002.
Wechter et al., Cancer Res., 57:4316-4324, 1997.
Wei et al., Gene Ther., 1(4):261-268, 1994.
Weinberger et al. Mol. Cell. Biol., 8:988, 1984.
Whitesell et al., Proc. Natl. Acad. Sci. USA, 91(18):8324-8328, 1994.
Williams, Sci STKE, 89:RE2, 2001.
Winde et al., Cancer Lett., 134(2): 201-7, 1998.
Winer et al., In: Cancer: Principles and Practice of Oncology, Devita et al., (Eds), Lippincott-Raven, 6^{th} Ed., 1651-1716, PA, 2000.

Wincott et al., Nucleic Acid. Res., 23:2677-2684, 1995.
Winoto and Baltimore, Cell, 59:649, 1989.
Wong et al., Gene, 10:87-94, 1980.
Wu et al., World J. Gastroenterol., 7:60-65, 2001.
Xia et al., Proc. Natl. Acad. Sci. USA., 103:7795-7800, 2006.
Yang and Huang, Gene Therapy, 4 (9):950-960, 1997.
Yang et al., J. Virol., 68(8):4847-4856, 1994.
Yoder et al., Blood, 82(Suppl.):347A, 1994.
You et al., Cell Growth Differ., 12:471-480, 2001.
You et al., Mol. Carcinogenesis, 33:228-236, 2002.
Yu et al., Cancer Res., 61:6569-6576, 2001.
Yutzeyet et al. Mol. Cell. Biol., 9:1397, 1989.
Zhang et al., J. Biol. Chem., 275:24436-24443, 2000.
Zhou et al., Exp. Hematol., 21(7):928-933, 1993.
Zhou et al., J. Exp. Med., 179(6):1867-1875, 1994.
Zhu et al., Science, 261(5118):209-211, 1993.

What is claimed is:

1. A method for treating cancer in a patient comprising
a) providing to the patient MDA-7; and
b) administering to the patient an EGFR inhibitor that is a small molecule tyrosine kinase inhibitor.

2. The method of claim 1, wherein the inhibitor is erlotinib, gefitinib, or lipatanib.

3. The method of claim 1, wherein the MDA-7 is provided to the patient by administering to the patient a composition comprising a nucleic acid having a sequence encoding MDA-7 polypeptide, wherein the MDA-7 polypeptide is expressed in the patient.

4. The method of claim 3, wherein the composition is a pharmaceutically acceptable composition.

5. The method of claim 3, wherein the nucleic acid is in a vector.

6. The method of claim 5, wherein the vector is a viral vector.

7. The method of claim 6, wherein about 10⁹ to about 10¹³ viral particles are administered to the patient/administration.

8. The method of claim 6, wherein the vector is an adenovirus vector.

9. The method of claim 8, wherein adenovirus vector is formulated with protamine.

10. The method of claim 3, wherein the MDA-7 nucleic acid composition is administered to the patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, intrathecally, orally, topically, locally, by inhalation, by injection, by infusion, by continuous infusion, by localized perfusion bathing target cells directly, via a catheter, or via a lavage.

11. The method of claim 3, wherein the MDA-7 nucleic acid composition comprises one or more lipids.

12. The method of claim 11, wherein the composition comprises DOTAP and cholesterol, or a derivative thereof.

13. The method of claim 1, wherein the MDA-7 is provided to the patient by administering to the patient a composition comprising purified MDA-7 protein.

14. The method of claim 13, wherein the purified MDA-7 protein composition is administered to the patient intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, intrathecally, orally, topically, locally, by inhalation, by injection, by infusion, by continuous infusion, by localized perfusion bathing target cells directly, via a catheter, or via a lavage.

15. The method of claim 1, wherein the MDA-7 is provided to the patient prior to administration of the inhibitor.

16. The method of claim 1, wherein the inhibitor is administered prior to providing the MDA-7 to the patient.

17. The method of claim 1, wherein the MDA-7 is provided to the patient via one route of administration and the inhibitor is administered to the patient via a different route.

18. The method of claim 1, wherein the MDA-7 is provided to the patient via the same route of administration as the inhibitor.

19. The method of claim 1, wherein the MDA-7 is provided to the patient with one week of administering the inhibitor.

20. The method of claim 1, wherein the MDA-7 is provided to the patient within 5 days of administering the inhibitor.

21. The method of claim 1, wherein the MDA-7 is provided to the patient within 48 hours of administering the inhibitor.

22. The method of claim 1, wherein the MDA-7 is provided to the patient within 24 hours of administering the inhibitor.

23. The method of claim 1, wherein MDA-7 is provided to the patient multiple times.

24. The method of claim 1, wherein the inhibitor is administered to the patient multiple times.

25. The method of claim 1, wherein the patient is given multiple courses of therapy with MDA-7 and the inhibitor.

26. The method of claim 1, further comprising treating the patient with another anti-cancer therapy.

27. The method of claim 26, wherein the anti-cancer therapy is chemotherapy or radiotherapy.

28. The method of claim 27, wherein the chemotherapy is platinum-based chemotherapy.

29. The method of claim 28, wherein the platinum-based chemotherapy involves one or more of carboplatin, paclitaxel, gemcitabine, or cisplatin.

30. The method of claim 27, wherein the chemotherapy involves more than one drug.

31. The method of claim 30, wherein the chemotherapy is the combination of carboplatin and paclitaxel or the combination of gemcitabine and cisplatin.

32. The method of claim 1, wherein the patient has or will undergo tumor resection.

33. The method of claim 1, further comprising identifying a patient in need of the treatment.

34. The method of claim 33, wherein identifying a patient in need of the treatment comprises taking a patient history regarding previous cancer treatment.

35. The method of claim 1, wherein the patient has failed previous cancer therapy or has a recurrent or metastasized cancer.

36. The method of claim 1, wherein the patient has cancer of the lung, prostate, liver, pancreas, bladder, breast, ovary, gastric, colon, head and neck, esophagus, synovium, brain, or bronchus.

37. A method of sensitizing a cancer cell to therapy with an EGFR inhibitor comprising providing an effective amount of MDA-7 and an EGFR inhibitor to the cell.

38. The method of claim 37, wherein the EGFR inhibitor is a tyrosine kinase inhibitor.

39. The method of claim 37, wherein the EGFR inhibitor is a biological agent that specifically targets EGFR.

40. The method of claim 39, wherein the inhibitor is panitumumab or matuxumab.

41. The method of claim 39, wherein the inhibitor is cetuximab.

42. The method of claim 37, wherein the MDA-7 is provided to cell before the EGFR inhibitor is provided to the cell.

43. A method of inducing apoptosis in a cancer cell comprising providing to the cancer cell and effective amount of a combination of MDA-7 and an EGFR inhibitor, wherein the cell undergoes apoptosis.

44. The method of claim 43, wherein the MDA-7 is provided to the cell by administering to the cell a nucleic acid encoding human MDA-7, wherein the nucleic acid is under the control of a heterologous promoter.

45. The method of claim 44, wherein the nucleic acid and EGFR inhibitor are provided to the cell at different times.

46. The method of claim 43, wherein the EGFR inhibitor is a small molecule tyrosine kinase inhibitor or biological agent that binds EGFR.
